(19) United States
(12) Patent Application Publication
(10) Pub. No.: US 2017/0015666 A1
(43) Pub. Date: Jan. 19, 2017

(54) ENHANCER OF ZESTE HOMOLOG 2 INHIBITORS

(71) Applicant: GLAXOSMITHKLINE INTELLECTUAL PROPERTY (NO.2) LIMITED, Brentford, Middlesex (GB)

(72) Inventors: William Henry MILLER, Collegeville, PA (US); Stuart Paul ROMERIL, Collegeville, PA (US); Xinrong TIAN, Collegeville, PA (US); Sharad Kumar VERMA, Collegeville, PA (US)

(21) Appl. No.: 15/123,769

(22) PCT Filed: Mar. 6, 2015
(86) PCT No.: PCT/IB2015/051649

§ 371 Date: Sep. 6, 2016

Related U.S. Application Data
(60) Provisional application No. 61/949,407, filed on Mar. 7, 2014.

(51) Int. Cl. C07D 471/14

(52) U.S. Cl. CPC C07D 471/14

(57) ABSTRACT

This invention relates to novel compounds according to Formula (I) which are inhibitors of Enhancer of Zeste Homolog 2 (EZH2), to pharmaceutical compositions containing them, to processes for their preparation, and to their use in therapy for the treatment of cancers.

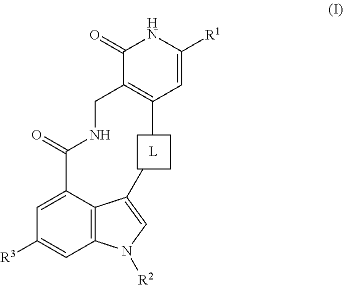

### FIELD OF THE INVENTION

This invention relates to compounds which inhibit Enhancer of Zeste Homolog 2 (EZH2) and thus are useful for inhibiting the proliferation of and/or inducing apoptosis in cancer cells.

### BACKGROUND OF THE INVENTION

Epigenetic modifications play an important role in the regulation of many cellular processes including cell proliferation, differentiation, and cell survival. Global epigenetic modifications are common in cancer, and include global changes in DNA and/or histone methylation, dysregulation of non-coding RNAs and nucleosome remodeling leading to aberrant activation or inactivation of oncogenes, tumor suppressors and signaling pathways. However, unlike genetic mutations which arise in cancer, these epigenetic changes can be reversed through selective inhibition of the enzymes involved. Several methylases involved in histone or DNA methylation are known to be dysregulated in cancer. Thus, selective inhibitors of particular methylases will be useful in the treatment of proliferative diseases such as cancer.

EZH2 (human EZH2 gene: Cardoso, C, et al; European J of Human Genetics, Vol. 8, No. 3 Pages 174-180, 2000) is the catalytic subunit of the Polycomb Repressor Complex 2 (PRC2) which functions to silence target genes by tri-methylating lysine 27 of histone H3 (H3K27me3). Histone H3 is one of the five main histone proteins involved in the structure of chromatin in eukaryotic cells. Featuring a main globular domain and a long N-terminal tail, Histones are involved with the structure of the nucleosomes, a ‘beads on a string’ structure. Histone proteins are highly post-translationally modified however Histone H3 is the most extensively modified of the five histones. The term “Histone H3” alone is purposely ambiguous in that it does not distinguish between sequence variants or modification state. Histone H3 is an important protein in the emerging field of epigenetics, where its sequence variants and variable modification states are thought to play a role in the dynamic and long term regulation of genes.

Increased EZH2 expression has been observed in numerous solid tumors including those of the prostate, breast, skin, bladder, liver, pancreas, head and neck and correlates with cancer aggressiveness, metastasis and poor outcome (Varambally et al., 2002; Kleer et al., 2003; Breuer et al., 2004; Bachmann et al., 2005; Weikert et al., 2005; Sudo et al., 2005; Bachmann et al., 2006). For instance, there is a greater risk of recurrence after prostatectomy in tumors expressing high levels of EZH2, increased metastasis, shorter disease-free survival and increased death in breast cancer patients with high EZH2 levels (Varambally et al., 2002; Kleer et al., 2003). More recently, inactivating mutations in UTX (ubiquitously transcribed tetratricopeptide repeats X), a H3K27 demethylase which functions in opposition to EZH2, have been identified in multiple solid and hematological tumor types (including renal, glioblastoma, esophageal, breast, colon, non-small cell lung, small cell lung, bladder, multiple myeloma, and chronic myeloid leukemia tumors), and low UTX levels correlate with poor survival in breast cancer suggesting that loss of UTX function leads to increased H3K27me3 and repression of target genes (Wang et al., 2010). Together, these data suggest that increased H3K27me3 levels contribute to cancer aggressiveness in many tumor types and that inhibition of EZH2 activity may provide therapeutic benefit.

Numerous studies have reported that direct knockdown of EZH2 via siRNA or shRNA or indirect loss of EZH2 via treatment with the SAH hydrolase inhibitor 3-deazaneplanocin A (DZNep) decreases cancer cell line proliferation and invasion in vitro and tumor growth in vivo (Gonzalez et al., 2008, GBM 2009). While the precise mechanism by which aberrant EZH2 activity leads to cancer progression is not known, many EZH2 target genes are tumor suppressors suggesting that loss of tumor suppressor function is a key mechanism. In addition, EZH2 overexpression in immortalized or primary epithelial cells promotes anchorage independent growth and invasion and requires EZH2 catalytic activity (Kleer et al., 2003; Cao et al., 2008).

Thus, there is strong evidence to suggest that inhibition of EZH2 activity decreases cellular proliferation and invasion. Accordingly, compounds that inhibit EZH2 activity would be useful for the treatment of cancer.

### SUMMARY OF THE INVENTION

The present invention relates to compounds according to Formula (I):

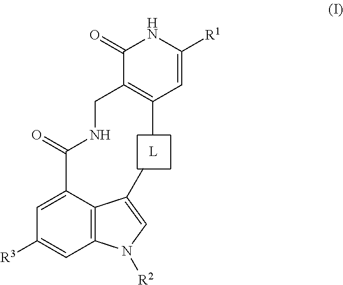

wherein:

L is (C₂-C₈)alkylenyl or (C₂-C₈)alkenylenyl;

R¹ is hydrogen, halogen, (C₁-C₆)alkyl, (C₂-C₆)alkenyl, (C₂-C₆)alkynyl, halo(C₁-C₄)alkyl, (C₃-C₆)cycloalkyl, (C₃-C₆)cycloalkyl(C₁-C₆)alkyl, (C₃-C₆)cycloalkyl(C₂-C₆)alkenyl, (C₅-C₆)cycloalkenyl, (C₅-C₆)cycloalkenyl(C₁-C₆)alkyl, (C₅-C₆)cycloalkenyl(C₂-C₆)alkenyl, (C₆-C₁₀)bicycloalkyl, heterocycloalkyl, heterocycloalkyl(C₁-C₆)alkyl-, heterocycloalkyl(C₂-C₆)alkenyl, phenyl, phenyl(C₁-C₆)alkyl, phenyl(C₂-C₆)alkenyl, heteroaryl, heteroaryl(C₁-C₆)alkyl, heteroaryl(C₂-C₆)alkenyl, cyano, —C(O)R^{a}, —CO₂R^{a}, —C(O)NR^{a}R^{b}, —C(O)NR^{a}NR^{a}R^{b}, —SR^{a}, —S(O)R^{a}, —SO₂R^{a}, —SO₂NR^{a}R^{b}, nitro, —NR^{a}R^{b}, R^{a}R^{b}N(C₁-C₄)alkyl-, —NR^{a}C(O)R^{b}, —NR^{a}C(O)NR^{a}R^{b}, —NR^{a}C(O)OR^{a}, —NR^{a}SO₂R^{b}, —R^{a}SO₂R^{a}R^{b}, —NR^{a}R^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, or —OC(O)NR^{a}R^{b}, wherein each cycloalkyl, cycloalkenyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂N^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;

R² is hydrogen, halogen, (C₁-C₆)alkyl, (C₂-C₆)alkenyl, (C₂-C₆)alkynyl, halo(C₁-C₄)alkyl, (C₃-C₆)cycloalkyl, (C₃-C₆)cycloalkyl(C₁-C₆)alkyl, (C₃-C₆)cycloalkyl(C₂-C₆)alkenyl, (C₅-C₆)cycloalkenyl, (C₅-C₆)cycloalkenyl(C₁-C₆)alkyl, (C₅-C₆)cycloalkenyl(C₂-C₆)alkenyl, (C₆-C₁₀)bicycloalkyl, heterocycloalkyl, heterocycloalkyl(C₁-C₆)alkyl-, heterocycloalkyl(C₂-C₆)alkenyl, phenyl, phenyl(C₁-C₆)alkyl, phenyl(C₂-C₆)alkenyl, heteroaryl, heteroaryl(C₁-C₆)alkyl, heteroaryl(C₂-C₆)alkenyl, —C(O)R^{a}, —CO₂R^{a}, —C(O)NR^{a}R^{b}, or —C(O)NR^{a}NR^{a}R^{b}, wherein each cycloalkyl, cycloalkenyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;

R³ is selected from the group consisting of hydrogen, halogen, (C₁-C₆)alkyl, (C₂-C₆)alkenyl, (C₂-C₆)alkynyl, (C₁-C₄)alkoxy, —B(OH)₂, (C₃-C₆)cycloalkyl, (C₃-C₆)cycloalkyl(C₁-C₄)alkyl-, (C₆-C₁₀)bicycloalkyl, heterocycloalkyl, heterocycloalkyl(C₁-C₄)alkyl-, phenyl, phenyl(C₁-C₂)alkyl, heteroaryl, heteroaryl(C₁-C₂)alkyl, cyano, —C(O)R^{a}, —CO₂R^{a}, —C(O)NR^{a}R^{b}, —C(O)NR^{a}NR^{a}R^{b}, —SR^{a}, —S(O)R^{a}, —SO₂R^{a}, —SO₂NR^{a}R^{b}, nitro, —NR^{a}R^{b}, R^{a}R^{b}N(C₁-C₄)alkyl-, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, R^{a}O(C₁-C₄)alkyl-, R^{a}O(C₃-C₆)alkynyl-, —OC(O)R^{a}, and —OC(O)NR^{a}R^{b}, wherein each cycloalkyl, bicycloalkyl, heterocycloalkyl, phenyl, or heteroaryl group is optionally substituted 1, 2, or 3 times, independently, by R^{c}—(C₁-C₆)alkyl-O—, R—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;

each R^{c} is independently —S(O)R^{a}, —SO₂R^{a}—NR^{a}R^{b}, —NR^{a}C(O)OR^{a}, —NR^{a}SO₂R^{b}, or —CO₂R^{a}; and

R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl (C₁-C₂)alkyl-, heteroaryl(C₁-C₄)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂;

or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring;

or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring;

or a pharmaceutically acceptable salt thereof.

Another aspect of this invention relates to a method of inducing apoptosis in cancer cells of solid tumors; treating solid tumor cancers.

Another aspect of the invention relates to pharmaceutical preparations comprising compounds of Formula (I) and pharmaceutically acceptable excipients.

In another aspect, there is provided the use of a compound of Formula (I) or a pharmaceutically acceptable salt or solvate thereof, in the preparation of a medicament for use in the treatment of a disorder mediated by EZH2, such as by inducing apoptosis in cancer cells.

In another aspect, this invention provides for the use of a compound of Formula (I) or a pharmaceutically acceptable salt thereof for the treatment of diseases mediated by EZH2. The invention further provides for the use of a compound of Formula (I) or a pharmaceutically acceptable salt thereof as an active therapeutic substance in the treatment of a disease mediated by EZH2.

In another aspect, the invention provides a compound of Formula (I) or a pharmaceutically acceptable salt thereof for use in therapy.

In another aspect, there is provided a compound of Formula (I) or a pharmaceutically acceptable salt thereof for use in the treatment of a disorder mediated by EZH2.

In another aspect, there is provided a compound of Formula (I) or a pharmaceutically acceptable salt thereof for use in the treatment of cellular proliferation diseases.

In another aspect, there is provided a compound of Formula (I) or a pharmaceutically acceptable salt thereof for use in the treatment of cancer, including the treatment of solid tumors, for example brain (gliomas), glioblastomas, leukemias, lymphomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, gastric, bladder, head and neck, kidney, lung, liver, melanoma, renal, ovarian, pancreatic, prostate, sarcoma, osteosarcoma, giant cell tumor of bone, and thyroid.

In another aspect there is provided methods of co-administering the presently invented compounds of Formula (I) with other active ingredients.

In another aspect there is provided a combination of a compound of Formula (I) or a pharmaceutically acceptable salt thereof and at least one anti-neoplastic agent for use in the treatment of a disorder mediated by EZH2.

In another aspect there is provided a combination of a compound of Formula (I) or a pharmaceutically acceptable salt thereof and at least one anti-neoplastic agent for use in the treatment of cellular proliferation diseases.

In another aspect there is provided a combination of a compound of Formula (I) or a pharmaceutically acceptable salt thereof and at least one anti-neoplastic agent for use in the treatment of cancer, including the treatment of solid tumors, for example brain (gliomas), glioblastomas, leukemias, lymphomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, gastric, bladder, head and neck, kidney, lung, liver, melanoma, renal, ovarian, pancreatic, prostate, sarcoma, osteosarcoma, giant cell tumor of bone, and thyroid.

### DETAILED DESCRIPTION OF THE INVENTION

This invention relates to compounds of the Formula (I) as defined above.

In one embodiment, this invention relates to compounds of Formula (I), wherein R¹ is hydrogen, halogen, (C₁-C₆)alkyl, halo(C₁-C₄)alkyl, (C₃-C₆)cycloalkyl, (C₃-C₆)cycloalkyl(C₁-C₄)alkyl, phenyl, or phenyl(C₁-C₂)alkyl. In another embodiment, this invention relates to compounds of Formula (I), wherein R¹ is (C₁-C₄)alkyl. In a specific embodiment, this invention relates to compounds of Formula (I), wherein R¹ is methyl.

In another embodiment, this invention relates to compounds of Formula (I), wherein R² is hydrogen, halogen, (C₁-C₆)alkyl, halo(C₁-C₄)alkyl, (C₃-C₆)cycloalkyl, (C₃-C₆)cycloalkyl(C₁-C₄)alkyl, phenyl, or phenyl(C₁-C₂)alkyl. In another embodiment, this invention relates to compounds of Formula (I), wherein R² is (C₁-C₄)alkyl. In a specific embodiment, this invention relates to compounds of Formula (I), wherein R² is isopropyl or s-butyl. In a more specific embodiment, this invention relates to compounds of Formula (I), wherein R² is isopropyl.

In another embodiment, this invention relates to compounds of Formula (I), wherein R³ is heteroaryl which is optionally substituted 1 or 2 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl; each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl(C₁-C₂)alkyl-, heteroaryl(C₁-C₂)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂; or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring; or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring.

In another embodiment, this invention relates to compounds of Formula (I), wherein R³ is heteroaryl which is optionally substituted by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl. In another embodiment, this invention relates to compounds of Formula (I), wherein R³ is heteroaryl which is optionally substituted by heterocycloalkyl or (C₁-C₄)alkyl-heterocycloalkyl-. In another embodiment, this invention relates to compounds of Formula (I), wherein R³ is heteroaryl which is optionally substituted by —NR^{a}R^{b}. In another embodiment, this invention relates to compounds of Formula (I), wherein R³ is furanyl, thiophenyl, thienyl, pyrrolyl, imidazolyl, pyrazolyl, triazolyl, tetrazolyl, thiazolyl, isothiazolyl, 1,2,3-triazolyl, 1,2,4-triazolyl, oxazolyl, isoxazolyl, 1,2,3-oxadiazolyl, 1,2,5-oxadiazolyl, thiadiazolyl, isothiazolyl, tetrazolyl, pyridinyl, pyridazinyl, pyrazinyl, pyrimidinyl, or triazinyl, each of which is optionally substituted by —NR^{a}R^{b}. In another embodiment, this invention relates to compounds of Formula (I), wherein R³ is furanyl, thiophenyl, thienyl, pyrrolyl, imidazolyl, pyrazolyl, triazolyl, tetrazolyl, thiazolyl, isothiazolyl, 1,2,3-triazolyl, 1,2,4-triazolyl, oxazolyl, isoxazolyl, 1,2,3-oxadiazolyl, 1,2,5-oxadiazolyl, thiadiazolyl, isothiazolyl, tetrazolyl, pyridinyl, pyridazinyl, pyrazinyl, pyrimidinyl, or triazinyl, each of which is optionally substituted by pyrrolidinyl, piperidinyl, piperazinyl, 4-methylpiperazinyl, morpholinyl, or thiomorpholinyl.

In another embodiment, this invention relates to compounds of Formula (I), wherein R³ is pyridinyl which is optionally substituted by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl; each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl(C₁-C₂)alkyl-, heteroaryl(C₁-C₂)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂; or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring; or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring.

In another embodiment, this invention relates to compounds of Formula (I), wherein R³ is pyridinyl which is optionally substituted by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl. In another embodiment, this invention relates to compounds of Formula (I), wherein R³ is pyridinyl which is optionally substituted by heterocycloalkyl or (C₁-C₄)alkyl-heterocycloalkyl-. In another embodiment, this invention relates to compounds of Formula (I), wherein R³ is pyridinyl which is optionally substituted by —NR^{a}R^{b}. In another embodiment, this invention relates to compounds of Formula (I), wherein R³ is pyridinyl which is optionally substituted by pyrrolidinyl, piperidinyl, piperazinyl, 4-methylpiperazinyl, morpholinyl, or thiomorpholinyl. In another embodiment, this invention relates to compounds of Formula (I), wherein R³ is pyridinyl which is substituted by piperazinyl.

In another embodiment, this invention relates to compounds of Formula (I), wherein R³ is selected from the group consisting of hydrogen, —SO₂(C₁-C₄)alkyl, halogen, (C₁-C₆)alkyl, (C₂-C₆)alkenyl, (C₂-C₆)alkynyl, hydroxy(C₁-C₄)alkyl-, hydroxy(C₃-C₆)alkynyl-, (C₁-C₄)alkoxy, phenyl, heteroaryl, and cyano, wherein said phenyl or heteroaryl group is optionally substituted 1 or 2 times, independently, by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl. In another embodiment, this invention relates to compounds of Formula (I), wherein R³ is selected from the group consisting of halogen, phenyl, and heteroaryl, wherein said phenyl or heteroaryl group is optionally substituted 1 or 2 times, independently, by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl. In another embodiment, this invention relates to compounds of Formula (I), wherein R³ is selected from the group consisting of halogen, phenyl, and heteroaryl, wherein said phenyl or heteroaryl group is optionally substituted by heterocycloalkyl or (C₁-C₄)alkyl-heterocycloalkyl-.

In another embodiment, this invention relates to compounds of Formula (I), wherein R³ is selected from the group consisting of hydrogen, cyano, halogen, (C₁-C₄)alkoxy, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, phenyl, pyridinyl, pyridazinyl, pyrazinyl, pyrimidinyl, and triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, phenyl, pyridinyl, pyridazinyl, pyrazinyl, pyrimidinyl, or triazinyl is optionally substituted by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl.

In another embodiment, this invention relates to compounds of Formula (I), wherein R³ is phenyl which is optionally substituted by —NR^{a}R^{b} or R^{a}R^{b}N(C₁-C₄)alkyl-.

In another embodiment, this invention relates to compounds of Formula (I), wherein R³ is cyano, halogen, (C₁-C₄)alkyl, (C₂-C₆)alkenyl, (C₂-C₆)alkynyl, hydroxy(C₁-C₄)alkyl-, hydroxy(C₃-C₆)alkynyl-, or (C₁-C₄)alkoxy. In another embodiment, this invention relates to compounds of Formula (I), wherein R³ is hydroxy(C₃-C₆)alkynyl-.

In another embodiment, this invention relates to compounds of Formula (I), wherein R³ is cyano, halogen, (C₁-C₄)alkyl, or (C₁-C₄)alkoxy. In another embodiment, this invention relates to compounds of Formula (I), wherein R³ is halogen. In a specific embodiment, this invention relates to compounds of Formula (I), wherein R³ is fluorine, chlorine, or bromine. In a more specific embodiment, this invention relates to compounds of Formula (I), wherein R³ is chlorine. In another specific embodiment, this invention relates to compounds of Formula (I), wherein R³ is cyano.

In another embodiment, this invention relates to compounds of Formula (I), wherein L is (C₄-C₅)alkylenyl or (C₄-C₅)alkenylenyl. In another embodiment, this invention relates to compounds of Formula (I), wherein L is selected from the group consisting of:

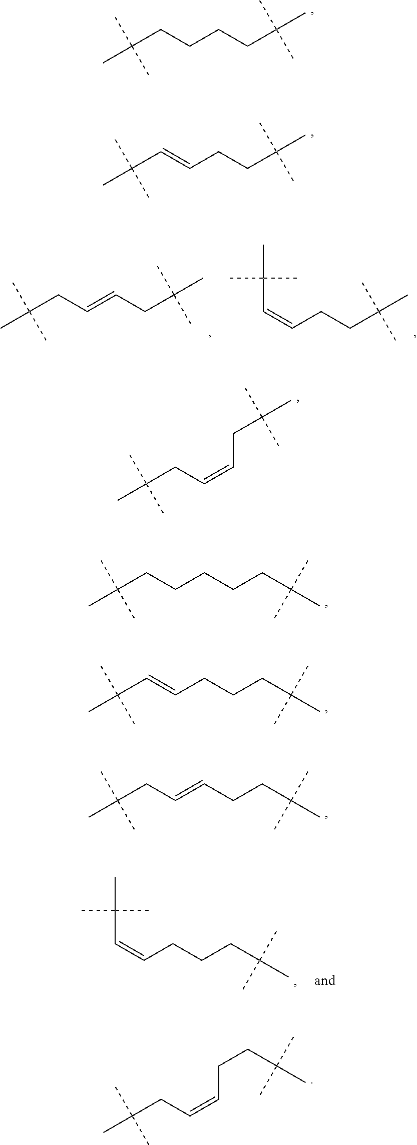

In another embodiment, this invention relates to compounds of Formula (I), wherein L is selected from the group consisting of:

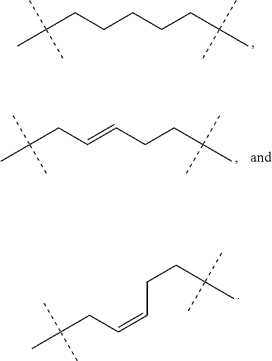

In another embodiment, this invention relates to compounds of Formula (I), wherein L is

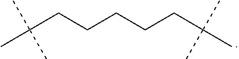

In another specific embodiment, this invention relates to compounds of Formula (I), wherein L is

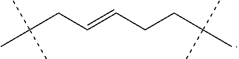

In another specific embodiment, this invention relates to compounds of Formula (I), wherein L is

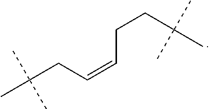

In a particular embodiment, this invention relates to compounds of Formula (I), wherein:

L is (C₄-C₅)alkylenyl or (C₄-C₅)alkenylenyl;

R¹ is hydrogen, halogen, (C₁-C₆)alkyl, halo(C₁-C₄)alkyl, (C₃-C₆)cycloalkyl, (C₃-C₆)cycloalkyl(C₁-C₄)alkyl, phenyl, or phenyl(C₁-C₂)alkyl;

R² is hydrogen, halogen, (C₁-C₆)alkyl, halo(C₁-C₄)alkyl, (C₃-C₆)cycloalkyl, (C₃-C₆)cycloalkyl(C₁-C₄)alkyl, phenyl, or phenyl(C₁-C₂)alkyl; and

R³ is selected from the group consisting of hydrogen, —SO₂(C₁-C₄)alkyl, halogen, (C₁-C₆)alkyl, (C₂-C₆)alkenyl, (C₂-C₆)alkynyl, hydroxy(C₁-C₄)alkyl-, hydroxy(C₃-C₆)alkynyl-, (C₁-C₄)alkoxy, phenyl, heteroaryl, and cyano, wherein said phenyl or heteroaryl group is optionally substituted 1 or 2 times, independently, by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl;

or a pharmaceutically acceptable salt thereof.

In another particular embodiment, this invention relates to compounds of Formula (I), wherein:

L is (C₄-C₅)alkylenyl or (C₄-C₅)alkenylenyl;

R¹ is (C₁-C₄)alkyl;

R² is (C₁-C₄)alkyl; and

R³ is selected from the group consisting of halogen, phenyl, and heteroaryl, wherein said phenyl or heteroaryl group is optionally substituted by heterocycloalkyl or (C₁-C₄)alkyl-heterocycloalkyl-;

or a pharmaceutically acceptable salt thereof.

In another particular embodiment, this invention relates to compounds of Formula (I), wherein:

L is selected from the group consisting of:

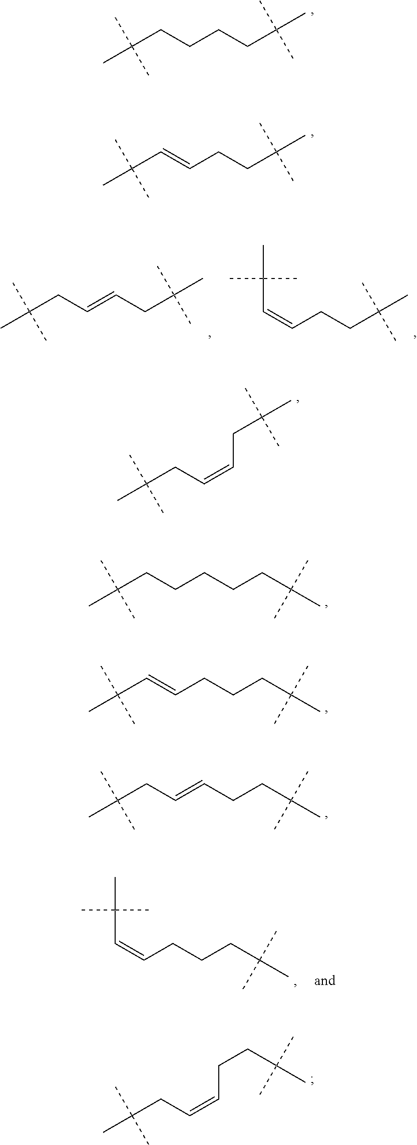

R¹ is (C₁-C₄)alkyl;

R² is (C₁-C₄)alkyl; and

R³ is halogen;

or a pharmaceutically acceptable salt thereof.

In another particular embodiment, this invention relates to compounds of Formula (I), wherein:

L is selected from the group consisting of:

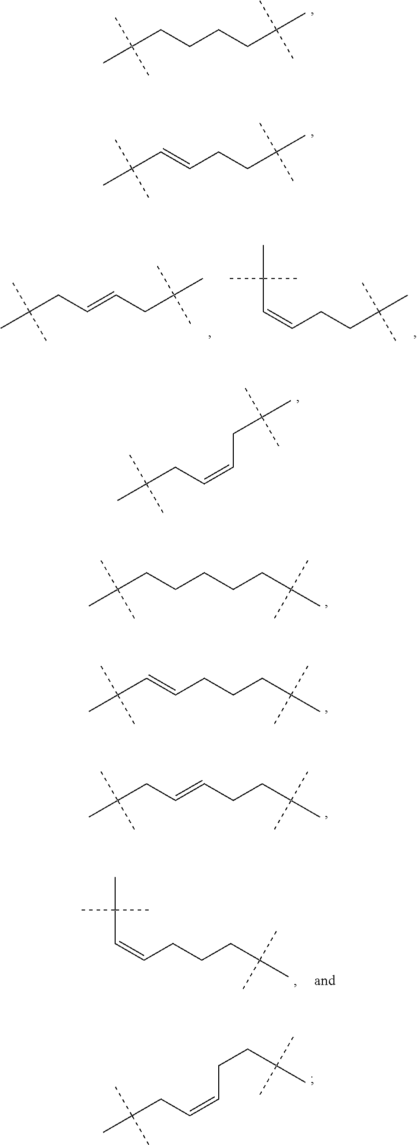

R¹ is (C₁-C₄)alkyl;

R² is (C₁-C₄)alkyl; and

R³ is pyridinyl which is optionally substituted by pyrrolidinyl, piperidinyl, piperazinyl, 4-methylpiperazinyl, morpholinyl, or thiomorpholinyl;

or a pharmaceutically acceptable salt thereof.

Specific compounds of this invention include:
(E)-2-chloro-4-isopropyl-12-methyl-9,10,15,16-tetrahydro-4H-pyrido[4′,3′:11,12][1]azacyclotridecino[5,4,3-cd]indole-14,17(6H,13H)-dione;
(Z)-2-chloro-4-isopropyl-12-methyl-9,10,15,16-tetrahydro-4H-pyrido[4′,3′:11,12][1]azacyclotridecino[5,4,3-cd]indole-14,17(6H,13H)-dione;
2-chloro-4-isopropyl-12-methyl-7,8,9,10,15,16-hexahydro-4H-pyrido[4′,3′:11,12][1]azacyclotridecino[5,4,3-cd]indole-14,17(6H,13H)-dione;
(E)-4-isopropyl-12-methyl-2-(6-(piperazin-1-yl)pyridin-3-yl)-9,10,15,16-tetrahydro-4H-pyrido[4′,3′:11,12][1]azacyclotridecino[5,4,3-cd]indole-14,17(6H,13H)-dione;
(E)-4-isopropyl-12-methyl-14,17-dioxo-6,9,10,13,14,15,16,17-octahydro-4H-pyrido[4′,3′:11,12][1]azacyclotridecino[5,4,3-cd]indole-2-carbonitrile; and
(E)-2-(3-hydroxy-3-methylbut-1-yn-1-yl)-4-isopropyl-12-methyl-9,10,15,16-tetrahydro-4H-pyrido[4′,3′:11,12][1]azacyclotridecino[5,4,3-cd]indole-14,17(6H,13H)-dione;

or pharmaceutically acceptable salts thereof.

Typically, but not absolutely, the salts of the present invention are pharmaceutically acceptable salts. Salts of the disclosed compounds containing a basic amine or other basic functional group may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycolates, tartrates mandelates, and sulfonates, such as xylenesulfonates, methanesulfonates, propanesulfonates, naphthalene-1-sulfonates and naphthalene-2-sulfonates.

Salts of the disclosed compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

Other salts, which are not pharmaceutically acceptable, may be useful in the preparation of compounds of this invention and these should be considered to form a further aspect of the invention. These salts, such as oxalic or trifluoroacetate, while not in themselves pharmaceutically acceptable, may be useful in the preparation of salts useful as intermediates in obtaining the compounds of the invention and their pharmaceutically acceptable salts.

The compound of Formula (I) or a salt thereof may exist in stereoisomeric forms (e.g., it contains one or more asymmetric carbon atoms). The individual stereoisomers (enantiomers and diastereomers) and mixtures of these are included within the scope of the present invention. Likewise, it is understood that a compound or salt of Formula (I) may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. It is to be understood that the present invention includes all combinations and subsets of the particular groups defined hereinabove.

The subject invention also includes isotopically-labeled compounds, which are identical to those recited in Formula (I) and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts thereof include isotopes of hydrogen, carbon, nitrogen, oxygen, phosphorous, sulfur, fluorine, chlorine, and iodine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I, and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of Formula (I) and following of this invention can generally be prepared by carrying out the procedures disclosed in the Schemes and/or in the Examples below, by substituting a readily available isotopically labeled reagent for a non-isotopically labeled reagent.

The invention further provides a pharmaceutical composition (also referred to as pharmaceutical formulation) comprising a compound of Formula (I) or pharmaceutically acceptable salt thereof and one or more excipients (also referred to as carriers and/or diluents in the pharmaceutical arts). The excipients are acceptable in the sense of being compatible with the other ingredients of the formulation and not deleterious to the recipient thereof (i.e., the patient).

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

Pharmaceutical compositions may be in unit dose form containing a predetermined amount of active ingredient per unit dose. Such a unit may contain a therapeutically effective dose of the compound of Formula (I) or salt thereof or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well-known in the pharmacy art.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).

When adapted for oral administration, pharmaceutical compositions may be in discrete units such as tablets or capsules; powders or granules; solutions or suspensions in aqueous or non-aqueous liquids; edible foams or whips; oil-in-water liquid emulsions or water-in-oil liquid emulsions. The compound or salt thereof of the invention or the pharmaceutical composition of the invention may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutical carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants such as colloidal silica, talc, magnesium stearate, calcium stearate, solid polyethylene glycol can be added to the powder mixture before the filling operation. A disintegrating or solubilizing agent such as agar-agar, calcium carbonate, or sodium carbonate can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars, such as glucose or beta-lactose, corn sweeteners, natural and synthetic gums such as acacia, tragacanth, sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, and aliginate, gelatin, or polyvinyl pyrrolidone, a solution retardant such as paraffin, a resorption accelerator such as a quaternary salt, and/or an absorption agent such as bentonite, kaolin, or dicalcium phosphate. The powder mixture can be granulated by wetting a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compound or salt of the present invention can also be combined with a free-flowing inert carrier and compressed into tablets directly without going through the granulating or slugging steps. A clear opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving the compound or salt thereof of the invention in a suitably flavoured aqueous solution, while elixirs are prepared through the use of a non-toxic alcoholic vehicle. Suspensions can be formulated by dispersing the compound or salt of the invention in a non-toxic vehicle. Solubilizers and emulsifiers, such as ethoxylated isostearyl alcohols and polyoxyethylene sorbitol ethers, preservatives, flavor additives such as peppermint oil, natural sweeteners, saccharin, or other artificial sweeteners, and the like, can also be added.

Where appropriate, dosage unit formulations for oral administration can be microencapsulated. The formulation can also be prepared to prolong or sustain the release as, for example, by coating or embedding particulate material in polymers, wax, or the like.

In the present invention, tablets and capsules are preferred for delivery of the pharmaceutical composition.

In accordance with another aspect of the invention there is provided a process for the preparation of a pharmaceutical composition comprising mixing (or admixing) a compound of Formula (I) or salt thereof with at least one excipient.

The present invention also provides a method of treatment in a mammal, especially a human. The compounds and compositions of the invention are used to treat cellular proliferation diseases. Disease states which can be treated by the methods and compositions provided herein include, but are not limited to, cancer (further discussed below), autoimmune disease, fungal disorders, arthritis, graft rejection, inflammatory bowel disease, proliferation induced after medical procedures, including, but not limited to, surgery, angioplasty, and the like. It is appreciated that in some cases the cells may not be in a hyper or hypo proliferation state (abnormal state) and still requires treatment. For example, during wound healing, the cells may be proliferating “normally”, but proliferation enhancement may be desired. Thus, in one embodiment, the invention herein includes application to cells or individuals afflicted or impending affliction with any one of these disorders or states.

The compositions and methods provided herein are particularly deemed useful for the treatment of cancer including tumors such as prostate, breast, brain, skin, cervical carcinomas, testicular carcinomas, etc. They are particularly useful in treating metastatic or malignant tumors. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one or related of the above identified conditions.

The instant compounds can be combined with or co-administered with other therapeutic agents, particularly agents that may enhance the activity or time of disposition of the compounds. Combination therapies according to the invention comprise the administration of at least one compound of the invention and the use of at least one other treatment method. In one embodiment, combination therapies according to the invention comprise the administration of at least one compound of the invention and surgical therapy. In one embodiment, combination therapies according to the invention comprise the administration of at least one compound of the invention and radiotherapy. In one embodiment, combination therapies according to the invention comprise the administration of at least one compound of the invention and at least one supportive care agent (e.g., at least one anti-emetic agent). In one embodiment, combination therapies according to the present invention comprise the administration of at least one compound of the invention and at least one other chemotherapeutic agent. In one particular embodiment, the invention comprises the administration of at least one compound of the invention and at least one anti-neoplastic agent. In yet another embodiment, the invention comprises a therapeutic regimen where the EZH2 inhibitors of this disclosure are not in and of themselves active or significantly active, but when combined with another therapy, which may or may not be active as a standalone therapy, the combination provides a useful therapeutic outcome.

By the term “co-administering” and derivatives thereof as used herein refers to either simultaneous administration or any manner of separate sequential administration of an EZH2 inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of specified cancers in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; DNA methyltransferase inhibitors such as azacitidine and decitabine; signal transduction pathway inhibitors; non-receptor tyrosine kinase angiogenesis inhibitors; immunotherapeutic agents; proapoptotic agents; and cell cycle signaling inhibitors.

Typically, any chemotherapeutic agent that has activity against a susceptible neoplasm being treated may be utilized in combination with the compounds the invention, provided that the particular agent is clinically compatible with therapy employing a compound of the invention. Typical anti-neoplastic agents useful in the present invention include, but are not limited to: alkylating agents, anti-metabolites, antitumor antibiotics, antimitotic agents, nucleoside analogues, topoisomerase I and II inhibitors, hormones and hormonal analogues; retinoids, histone deacetylase inhibitors; signal transduction pathway inhibitors including inhibitors of cell growth or growth factor function, angiogenesis inhibitors, and serine/threonine or other kinase inhibitors; cyclin dependent kinase inhibitors; antisense therapies and immunotherapeutic agents, including monoclonals, vaccines or other biological agents.

Nucleoside analogues are those compounds which are converted to deoxynucleotide triphosphates and incorporated into replicating DNA in place of cytosine. DNA methyltransferases become covalently bound to the modified bases resulting in an inactive enzyme and reduced DNA methylation. Examples of nucleoside analogues include azacitidine and decitabine which are used for the treatment of myelodysplastic disorder. Histone deacetylase (HDAC) inhibitors include vorinostat, for the treatment of cutaneous T-cell lymphoma. HDACs modify chromatin through the deacetylation of histones. In addition, they have a variety of substrates including numerous transcription factors and signaling molecules. Other HDAC inhibitors are in development.

Signal transduction pathway inhibitors are those inhibitors which block or inhibit a chemical process which evokes an intracellular change. As used herein this change is cell proliferation or differentiation or survival. Signal transduction pathway inhibitors useful in the present invention include, but are not limited to, inhibitors of receptor tyrosine kinases, non-receptor tyrosine kinases, SH2/SH3 domain blockers, serine/threonine kinases, phosphatidyl inositol-3-OH kinases, myoinositol signaling, and Ras oncogenes. Signal transduction pathway inhibitors may be employed in combination with the compounds of the invention in the compositions and methods described above.

Receptor kinase angiogenesis inhibitors may also find use in the present invention. Inhibitors of angiogenesis related to VEGFR and TIE-2 are discussed above in regard to signal transduction inhibitors (both are receptor tyrosine kinases). Other inhibitors may be used in combination with the compounds of the invention. For example, anti-VEGF antibodies, which do not recognize VEGFR (the receptor tyrosine kinase), but bind to the ligand; small molecule inhibitors of integrin (alphaᵥ beta₃) that inhibit angiogenesis; endostatin and angiostatin (non-RTK) may also prove useful in combination with the compounds of the invention. One example of a VEGFR antibody is bevacizumab (AVASTIN®).

Several inhibitors of growth factor receptors are under development and include ligand antagonists, antibodies, tyrosine kinase inhibitors, anti-sense oligonucleotides and aptamers. Any of these growth factor receptor inhibitors may be employed in combination with the compounds of the invention in any of the compositions and methods/uses described herein. Trastuzumab (Herceptin®) is an example of an anti-erbB2 antibody inhibitor of growth factor function. One example of an anti-erbB1 antibody inhibitor of growth factor function is cetuximab (Erbitux™, C225). Bevacizumab (Avastin®) is an example of a monoclonal antibody directed against VEGFR. Examples of small molecule inhibitors of epidermal growth factor receptors include but are not limited to lapatinib (Tykerb®) and erlotinib (TARCEVA®). Imatinib mesylate (GLEEVEC®) is one example of a PDGFR inhibitor. Examples of VEGFR inhibitors include pazopanib (Votrient®), ZD6474, AZD2171, PTK787, sunitinib and sorafenib.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325 (1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Int. Med., 111:273, 1989) and for the treatment of breast cancer (Holmes et al., J. Nat. Cancer Inst., 83:1797, 1991.). It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide. 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also known as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leukopenialeukopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leukopenialeukopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2(1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leukopenialeukopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leukopenialeukopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leukopenialeukopenia, thrombocytopenia, and anemia, is the most common dose limiting side effect of gemcitabine administration.

Methotrexate, N-[4 [[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leukopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I—DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I—DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the Formula (I), depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association a compound of formal (I) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of a compound of Formula (I). Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit pharmaceutical compositions for oral administration can be microencapsulated. The formulation can also be prepared to prolong or sustain the release as for example by coating or embedding particulate material in polymers, wax or the like.

Pharmaceutical compositions adapted for rectal administration may be presented as suppositories or as enemas.

Pharmaceutical compositions adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The pharmaceutical compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the pharmaceutical compositions may include other agents conventional in the art having regard to the type of formulation in question, for example those suitable for oral administration may include flavoring agents.

A therapeutically effective amount of a compound of the present invention will depend upon a number of factors including, for example, the age and weight of the intended recipient, the precise condition requiring treatment and its severity, the nature of the formulation, and the route of administration, and will ultimately be at the discretion of the attendant prescribing the medication. However, an effective amount of a compound of Formula (I) for the treatment of anemia will generally be in the range of 0.001 to 100 mg/kg body weight of recipient per day, suitably in the range of 0.01 to 10 mg/kg body weight per day. For a 70 kg adult mammal, the actual amount per day would suitably be from 7 to 700 mg and this amount may be given in a single dose per day or in a number (such as two, three, four, five or six) of sub-doses per day such that the total daily dose is the same. An effective amount of a salt or solvate, etc., may be determined as a proportion of the effective amount of the compound of Formula (I) per se. It is envisaged that similar dosages would be appropriate for treatment of the other conditions referred to above.

### DEFINITIONS

Terms are used within their accepted meanings. The following definitions are meant to clarify, but not limit, the terms defined.

As used herein, the term “alkyl” represents a saturated, straight or branched hydrocarbon moiety having the specified number of carbon atoms. The term “(C₁-C₆)alkyl” refers to an alkyl moiety containing from 1 to 6 carbon atoms. Exemplary alkyls include, but are not limited to methyl, ethyl, n-propyl, isopropyl, n-butyl, isobutyl, s-butyl, t-butyl, pentyl, and hexyl.

As used herein, the term “alkylenyl” represents a saturated, straight or branched divalent hydrocarbon radical having the specified number of carbon atoms. The term “(C₂-C₈)alkylenyl” refers to an alkylenyl moiety containing from 2 to 8 carbon atoms.

When the term “alkyl” is used in combination with other substituent groups, such as “halo(C₁-C₄)alkyl”, “hydroxy(C₁-C₄)alkyl” or “phenyl(C₁-C₂)alkyl-”, the term “alkyl” is intended to encompass a divalent straight or branched-chain hydrocarbon radical, wherein the point of attachment is through the alkyl moiety. The term “halo(C₁-C₄)alkyl” is intended to mean a radical having one or more halogen atoms, which may be the same or different, at one or more carbon atoms of an alkyl moiety containing from 1 to 4 carbon atoms, which is a straight or branched-chain carbon radical. Examples of “halo(C₁-C₄)alkyl” groups useful in the present invention include, but are not limited to, —CF₃ (trifluoromethyl), —CCl₃ (trichloromethyl), 1,1-difluoroethyl, 2,2,2-trifluoroethyl, and hexafluoroisopropyl. Examples of “phenyl(C₁-C₂)alkyl-” groups useful in the present invention include, but are not limited to, benzyl (phenylmethyl), 1-methylbenzyl (1-phenylethyl), and phenethyl (2-phenylethyl). Examples of “hydroxy(C₁-C₄)alkyl” groups useful in the present invention include, but are not limited to, hydroxymethyl, hydroxyethyl, and hydroxyisopropyl.

“Alkoxy” refers to a group containing an alkyl radical, defined hereinabove, attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups useful in the present invention include, but are not limited to, methoxy, ethoxy, n-propoxy, isopropoxy, n-butoxy, s-butoxy, isobutoxy, and t-butoxy.

When the term “alkenyl” is used it refers to straight or branched hydrocarbon chains containing the specified number of carbon atoms and at least 1 and up to 4 carbon-carbon double bonds. Examples include ethenyl (or ethenylene) and propenyl (or propenylene).

When the term “alkenylenyl” is used it refers to a straight or branched divalent hydrocarbon radical containing the specified number of carbon atoms and at least 1 and up to 4 carbon-carbon double bonds.

When the term “alkynyl” (or “alkynylene”) is used it refers to straight or branched hydrocarbon chains containing the specified number of carbon atoms and at least 1 and up to 4 carbon-carbon triple bonds. Examples include ethynyl (or ethynylene) and propynyl (or propynylene).

When “cycloalkyl” is used it refers to a non-aromatic, saturated, cyclic hydrocarbon ring containing the specified number of carbon atoms. So, for example, the term “(C₃-C₈)cycloalkyl” refers to a non-aromatic cyclic hydrocarbon ring having from three to eight carbon atoms. Exemplary “(C₃-C₈)cycloalkyl” groups useful in the present invention include, but are not limited to, cyclopropyl, cyclobutyl, cyclopentyl, cyclohexyl, cycloheptyl, and cyclooctyl.

As used herein, the term “cycloalkenyl” refers to a non-aromatic, cyclic hydrocarbon ring containing the specified number of carbon atoms and at least one carbon-carbon double bond. The term “(C₅-C₈)cycloalkenyl” refers to a non-aromatic cyclic hydrocarbon ring having from five to eight ring carbon atoms. Exemplary “(C₅-C₈)cycloalkenyl” groups useful in the present invention include cyclopentenyl, cyclohexenyl, cycloheptenyl, and cyclooctenyl.

As used herein, the term “cycloalkyloxy-” refers to a group containing a cycloalkyl radical, defined hereinabove, attached through an oxygen linking atom. Exemplary “(C₃-C₈)cycloalkyloxy-” groups useful in the present invention include cyclopropyloxy, cyclobutyloxy, cyclopentyloxy, cyclohexyloxy, cycloheptyloxy, and cyclooctyloxy.

As used herein, the term “bicycloalkyl” refers to a saturated, bridged, fused, or spiro, bicyclic hydrocarbon ring system containing the specified number of carbon atoms. Exemplary “(C₆-C₁₀)bicycloalkyl” groups include, but are not limited to bicyclo[2.1.1]hexyl, bicyclo[2.1.1]heptyl, bicyclo[3.2.1]octyl, bicyclo[2.2.2]octyl, bicyclo[3.2.2]nonyl, bicyclo[3.3.1]nonyl, bicyclo[3.3.2]decyl, bicyclo[4.3.1]decyl, bicyclo[2.2.0]hexyl, bicyclo[3.1.0]hexyl, bicyclo[3.2.0]heptyl, bicyclo[4.1.0]heptyl, octahydropentalenyl, bicyclo[4.2.0]octyl, decahydronaphthalenyl, spiro[3.3]heptyl, spiro[2.4]heptyl, spiro[3.4]octyl, spiro[2.5]octyl, spiro[4.4]nonyl, spiro[3.5]nonyl, and spiro[4.5]decyl.

The terms “halogen” and “halo” represent chloro, fluoro, bromo, or iodo substituents. “Hydroxy” or “hydroxyl” is intended to mean the radical —OH.

“Heterocycloalkyl” represents a group or moiety comprising a non-aromatic, monovalent monocyclic or bicyclic radical, which is saturated or partially unsaturated, containing 3 to 10 ring atoms, which includes 1 to 3 heteroatoms independently selected from nitrogen, oxygen and sulfur, including N-oxides, sulfur oxides, and dioxides. Illustrative examples of heterocycloalkyls useful in the present invention include, but are not limited to, aziridinyl, azetidinyl, pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, 1,3-dioxanyl, 1,4-dioxanyl, 1,3-oxathiolanyl, 1,3-oxathianyl, 1,3-dithianyl, 1,4-dithianyl, hexahydro-1H-1,4-diazepinyl, azabicylo[3.2.1]octyl, azabicylo[3.3.1]nonyl, azabicylo[4.3.0]nonyl, oxabicylo[2.2.1]heptyl, 1,1-dioxidotetrahydro-2H-thiopyranyl, and 1,5,9-triazacyclododecyl.

As used herein, the term “heteroaryl” refers to an aromatic ring system containing carbon(s) and at least one heteroatom selected from nitrogen, oxygen and sulfur, including N-oxides. Heteroaryl may be monocyclic or polycyclic, substituted or unsubstituted. A monocyclic heteroaryl group may have 1 to 4 heteroatoms in the ring, while a polycyclic heteroaryl may contain 1 to 8 heteroatoms. Bicyclic heteroaryl rings may contain from 8 to 10 member atoms. Monocyclic heteroaryl rings may contain from 5 to 6 member atoms (carbons and heteroatoms). Exemplary 5- to 6-membered heteroaryls include, but are not limited to, furanyl, thiophenyl, thienyl, pyrrolyl, imidazolyl, pyrazolyl, triazolyl, tetrazolyl, thiazolyl, isothiazolyl, 1,2,3-triazolyl, 1,2,4-triazolyl, oxazolyl, isoxazolyl, 1,2,3-oxadiazolyl, 1,2,5-oxadiazolyl, thiadiazolyl, isothiazolyl, tetrazolyl, pyridinyl, pyridazinyl, pyrazinyl, pyrimidinyl, and triazinyl. Other exemplary heteroaryl groups include, but are not limited to benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

As used herein, the term “cyano” refers to the group —CN.

As used herein, the term “optionally” means that the subsequently described event(s) may or may not occur, and includes both event(s) that occur and event(s) that do not occur.

As used herein, unless otherwise defined, the phrase “optionally substituted” or variations thereof denote an optional substitution, including multiple degrees of substitution, with one or more substituent group. The phrase should not be interpreted as duplicative of the substitutions herein described and depicted.

As used herein, the term “treatment” refers to alleviating the specified condition, eliminating or reducing one or more symptoms of the condition, slowing or eliminating the progression of the condition, and delaying the reoccurrence of the condition in a previously afflicted or diagnosed patient or subject.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula (I), as well as salts thereof, may be administered as the raw chemical. Additionally, the active ingredient may be presented as a pharmaceutical composition.

### Compound Preparation

### ABBREVIATIONS

| | |
|---|---|
| Boc₂O | di-tert-butyl dicarbonate |
| CaCl₂ | calcium chloride |
| Cbz | carboxybenzyl |
| CHCl₃ | chloroform |
| CH₃CN | acetonitrile |
| Cs₂CO₃ | cesium carbonate |
| DBU | 1,8-diazabicyclo[5.4.0]undec-7-ene |
| DCM | dichloromethane |
| DIAD | diisopropyl azodicarboxylate |
| DME | 1,2-dimethoxyethane |
| DMF | N,N-dimethylformamide |
| DMSO | dimethylsulfoxide |
| dppf | 1,1′-bis(diphenylphosphino)ferrocene |
| EtOAc | ethyl acetate |
| EDC | N-(3-dimethylaminopropyl)-N′-ethylcarbodiimide |
| | hydrochloride |
| ES | electrospray |
| Et₃N | triethylamine |
| Et₂O | diethyl ether |
| EtOH | ethanol |
| h | hour(s) |
| HCl | hydrochloric acid |
| H₂O | water |
| HOAt | 1-hydroxy-7-azabenzotriazole |
| HPLC | high-performance liquid chromatography |
| Hunig's base | N,N-diisopropylethylamine |
| LCMS | liquid chromatography mass spectrometry |
| MeOH | methanol |
| MgCl₂ | magnesium chloride |
| MgSO₄ | magnesium sulfate |
| min | minute(s) |
| MS | mass spectrometry |
| Na₂CO₃ | sodium carbonate |
| NaH | sodium hydride |
| NaHCO₃ | sodium bicarbonate |
| NaI | sodium iodide |
| NaOH | sodium hydroxide |
| Na₂SO₄ | sodium sulphate |
| NH₄Cl | ammonium chloride |
| PdCl₂(dppf)-DCM | [1,1′-bis(diphenylphosphino)ferrocene] |
| | dichloropalladium(II)-dichloromethane complex |
| Pd₂(dba)₃ | tris(dibenzylideneacetone)dipalladium(0) |
| Pd(PPh₃)₄ | tetrakis(triphenylphosphine)palladium(0) |
| TBME | tert-butyl methyl ether |
| TFA | trifluoroacetic acid |
| THF | tetrahydrofuran |

### Generic Synthesis Schemes

The compounds of this invention may be made by a variety of methods, including well-known standard synthetic methods. Illustrative general synthetic methods are set out below and then specific compounds of the invention are prepared in the working examples. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. In all of the schemes described below, protecting groups for sensitive or reactive groups are employed where necessary in accordance with general principles of synthetic chemistry. Protecting groups are manipulated according to standard methods of organic synthesis (T. W. Green and P. G. M. Wuts, (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention. Starting materials are commercially available or are made from commercially available starting materials using methods known to those skilled in the art.

The compounds of Formula (I) wherein L is alkenylenyl can be prepared according to Scheme 1 or by analogous methods. An appropriately functionalized alkenyl-substituted 4-carboxyindole A is coupled to an appropriately functionalized alkenyl-substituted 3-aminomethylpyridine B using an appropriate reagent, such as EDC, in an appropriate solvent, such as dimethyl sulfoxide. Ring closing metathesis of amide C using an appropriate reagent, such as the Grubbs first or second generation RCM catalyst, in an appropriate solvent, such as dichloromethane, provides macrocycle D. Hydrolysis of the 2-methoxypyridine moiety using an appropriate reagent, such as hydrochloric acid, in an appropriate solvent, such as 1,4-dioxane, affords compounds of Formula (I).

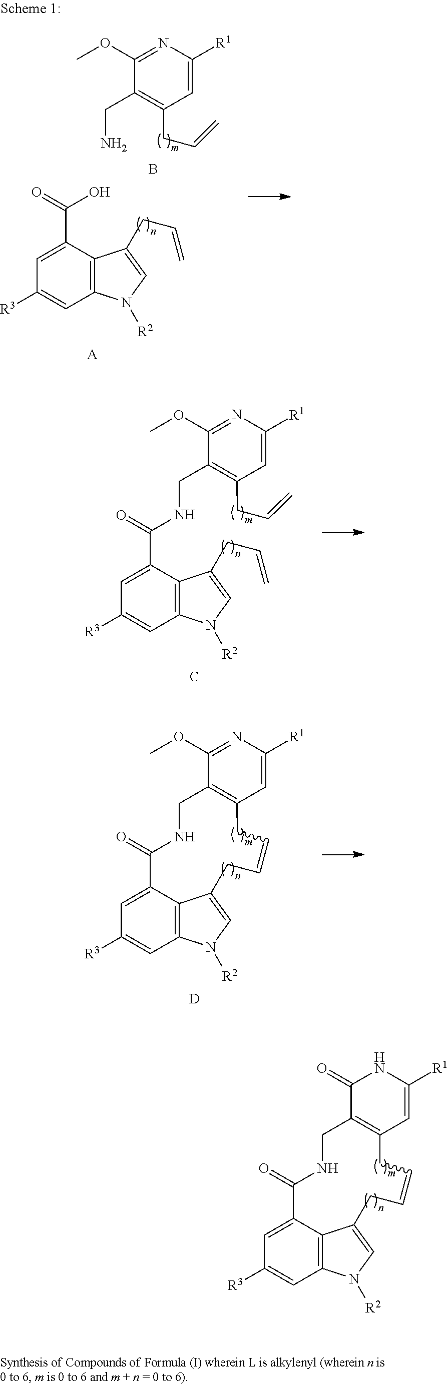

The compounds of Formula (I) wherein L is alkylenyl can be prepared according to Scheme 2 or by analogous methods. Hydrogenation of macrocycle D under appropriate conditions, such as in the presence of catalytic platinum on carbon in a hydrogen gas atmosphere, in an appropriate solvent, such as ethyl acetate, provides the saturated macrocycle E. Hydrolysis of the 2-methoxypyridine moiety using an appropriate reagent, such as hydrochloric acid, in an appropriate solvent, such as 1,4-dioxane, affords compounds of Formula (I).

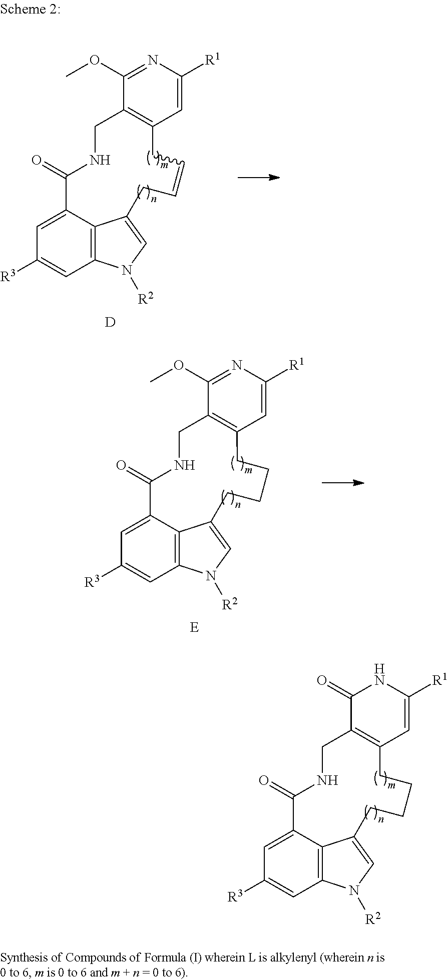

### EXPERIMENTALS

The following guidelines apply to all experimental procedures described herein. All reactions were conducted under a positive pressure of nitrogen using oven-dried glassware, unless otherwise indicated. Temperatures designated are external (i.e. bath temperatures), and are approximate. Air and moisture-sensitive liquids were transferred via syringe. Reagents were used as received. Solvents utilized were those listed as “anhydrous” by vendors. Molarities listed for reagents in solutions are approximate, and were used without prior titration against a corresponding standard. All reactions were agitated by stir bar, unless otherwise indicated. Heating was conducted using heating baths containing silicon oil, unless otherwise indicated. Reactions conducted by microwave irradiation (0-400 W at 2.45 GHz) were done so using a Biotage Initiator™ 2.0 instrument with Biotage microwave EXP vials (0.2-20 mL) and septa and caps. Irradiation levels utilized (i.e. high, normal, low) based on solvent and ionic charge were based on vendor specifications. Cooling to temperatures below −70° C. was conducted using dry ice/acetone or dry ice/2-propanol. Magnesium sulfate and sodium sulfate used as drying agents were of anhydrous grade, and were used interchangeably. Solvents described as being removed “in vacuo” or “under reduced pressure” were done so by rotary evaporation.

Preparative normal phase silica gel chromatography was carried out using either a Teledyne ISCO CombiFlash Companion instrument with RediSep or ISCO Gold silica gel cartridges (4 g-330 g), or an Analogix IF280 instrument with SF25 silica gel cartridges (4 g-300 g), or a Biotage SP1 instrument with HP silica gel cartridges (10 g-100 g). Purification by reverse phase HPLC was conducted using a YMC-pack column (ODS-A 75×30 mm) as solid phase, unless otherwise noted. A mobile phase of 25 mL/min A (acetonitrile-0.1% TFA): B (water-0.1% TFA), 10-80% gradient A (10 min) was utilized with UV detection at 214 nM, unless otherwise noted.

A PE Sciex API 150 single quadrupole mass spectrometer (PE Sciex, Thornhill, Ontario, Canada) was operated using electrospray ionization in the positive ion detection mode. The nebulizing gas was generated from a zero air generator (Balston Inc., Haverhill, Mass., USA) and delivered at 65 psi and the curtain gas was high purity nitrogen delivered from a Dewar liquid nitrogen vessel at 50 psi. The voltage applied to the electrospray needle was 4.8 kV. The orifice was set at 25 V and mass spectrometer was scanned at a rate of 0.5 scan/sec using a step mass of 0.2 amu and collecting profile data.

Method A LCMS. Samples were introduced into the mass spectrometer using a CTC PAL autosampler (LEAP Technologies, Carrboro, N.C.) equipped with a hamilton 10 uL syringe which performed the injection into a Valco 10-port injection valve. The HPLC pump was a Shimadzu LC-10ADvp (Shimadzu Scientific Instruments, Columbia, Md.) operated at 0.3 mL/min and a linear gradient 4.5% A to 90% B in 3.2 min. with a 0.4 min. hold. The mobile phase was composed of 100% (H₂O 0.02% TFA) in vessel A and 100% (CH₃CN 0.018% TFA) in vessel B. The stationary phase is Aquasil (C18) and the column dimensions were 1 mm×40 mm. Detection was by UV at 214 nm, evaporative light-scattering (ELSD) and MS.

Method B, LCMS. Alternatively, an Agilent 1100 analytical HPLC system with an LC/MS was used and operated at 1 mL/min and a linear gradient 5% A to 100% B in 2.2 min with a 0.4 min hold. The mobile phase was composed of 100% (H₂O 0.02% TFA) in vessel A and 100% (CH₃CN 0.018% TFA) in vessel B. The stationary phase was Zobax (C8) with a 3.5 um partical size and the column dimensions were 2.1 mm×50 mm. Detection was by UV at 214 nm, evaporative light-scattering (ELSD) and MS.

Method C, LCMS. Alternatively, an MDSSCIEX API 2000 equipped with a capillary column of (50×4.6 mm, 5 μm) was used. HPLC was done on Agilent-1200 series UPLC system equipped with column Zorbax SB-C18 (50×4.6 mm, 1.8 μm) eluting with CH₃CN: ammonium acetate buffer. The reactions were performed in the microwave (CEM, Discover).

¹H-NMR spectra were recorded at 400 MHz using a Bruker AVANCE 400 MHz instrument, with ACD Spect manager v. 10 used for reprocessing. Multiplicities indicated are: s=singlet, d=doublet, t=triplet, q=quartet, quint=quintet, sxt=sextet, m=multiplet, dd=doublet of doublets, dt=doublet of triplets etc. and br indicates a broad signal. All NMRs in DMSO-d₆ unless otherwise noted.

Analytical HPLC: Products were analyzed by Agilent 1100 Analytical Chromatography system, with 4.5×75 mm Zorbax XDB-C18 column (3.5 um) at 2 mL/min with a 4 min gradient from 5% CH₃CN (0.1% formic acid) to 95% CH₃CN (0.1% formic acid) in H₂O (0.1% formic acid) and a 1 min hold.

### PREPARATION OF EXAMPLES

### Example 1

### (E)-2-chloro-4-isopropyl-12-methyl-9,10,15,16-tetrahydro-4H-pyrido[4′,3′:11,12][1]azacyclotridecino[5,4,3-cd]indole-14,17(6H,13H)-dione

### Preparation of 4-(but-3-en-1-yl)-2-methoxy-6-methylnicotinonitrile

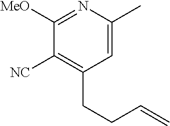

To a solution of 2-methoxy-4,6-dimethylnicotinonitrile (1.5 g, 9.25 mmol) in tetrahydrofuran (40 mL) was added lithium bis(trimethylsilyl)amide (10.17 mL, 10.17 mmol) at −78° C., and the mixture was stirred at −78° C. for 1 hour. 3-Bromoprop-1-ene (0.880 mL, 10.17 mmol) was added and the mixture was stirred at −78° C. for 1 hour and warmed to 0° C. over 1 hour. The mixture was then stirred at 0° C. for 3 hours. The reaction was quenched with saturated aqueous NH₄Cl solution and extracted with EtOAc (3×). The combined organics were dried over Na₂SO₄ and concentrated. The residue was purified using reverse HPLC using Trilution software, with a phenomenex Gemini 5u C18(2) 100 A, AXIA 30×100 mm 5 micron, 10-minute run (30 mL/min, 40% ACN/H₂O, 0.1% formic acid to 80% ACN/H₂O, 0.1% formic acid) with UV detection at 254 nm to afford 4-(but-3-en-1-yl)-2-methoxy-6-methylnicotinonitrile (1.01 g, 54%) as a pale yellow oil. LC-MS(ES) m/z=203 [M+H]⁺.

### Preparation of (4-(but-3-en-1-yl)-2-methoxy-6-methylpyridin-3-yl)methanamine

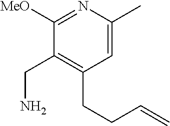

To a solution of 4-(but-3-en-1-yl)-2-methoxy-6-methylnicotinonitrile (700 mg, 3.46 mmol) in diethyl ether (15 mL) at 0° C. was added LAH (2 M in THF, 3.46 mL, 6.92 mmol), and the mixture was slowly warmed to room temperature and stirred at room temperature for 3 hours. The mixture was cooled with ice-bath and quenched with minimum amount of water (until no more hydrogen was generated). The mixture was treated with DCM and filtered, and the residue was washed with DCM:MeOH (10:1). The combined organic phases were concentrated, and the residue was purified using column chromatography (silica gel, 0 to 13% MeOH in DCM) to afford (4-(but-3-en-1-yl)-2-methoxy-6-methylpyridin-3-yl)methanamine (604 mg, 85%) as a pale yellow oil. LC-MS(ES) m/z=190 (major) 207 [M+H]⁺ (minor).

### Preparation of methyl 3-allyl-6-chloro-1H-indole-4-carboxylate

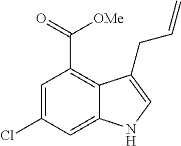 [SMILES: C=CCc1c[nH]c2cc(Cl)cc(C(=O)OC)c12]

To a solution of methyl 6-chloro-1H-indole-4-carboxylate (1000 mg, 4.77 mmol) in tetrahydrofuran (20 mL) was added allyl alcohol (0.324 mL, 4.77 mmol), and the mixture was degassed for 5 minutes by bubbling nitrogen. Triethylborane (1.431 mL, 1.431 mmol) and tetrakis(triphenylphosphine)palladium(0) (276 mg, 0.239 mmol) were added and the mixture was heated to 75° C. and stirred at 75° C. for 18 hours. The mixture was concentrated and the residue was purified using column chromatography (silica gel, 0 to 70% EtOAc in hexanes) to afford methyl 3-allyl-6-chloro-1H-indole-4-carboxylate (714 mg, 60%) as a white solid. LC-MS(ES) m/z=250 [M+H]⁺.

### Preparation of methyl 3-allyl-6-chloro-1-isopropyl-1H-indole-4-carboxylate

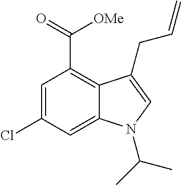 [SMILES: C=CCc1cn(C(C)C)c2cc(Cl)cc(C(=O)OC)c12]

To a solution of methyl 3-allyl-6-chloro-1H-indole-4-carboxylate (700 mg, 2.80 mmol) in N,N-dimethylformamide (10 mL) was added sodium hydride (60 wt % dispersion in mineral oil, 168 mg, 4.21 mmol), and the mixture was stirred at 0° C. for 30 minutes. 2-Iodopropane (620 mg, 3.64 mmol) was added and the reaction was stirred at room temperature for 18 hours. The mixture was quenched with 10% NH₄Cl aqueous solution and extracted with EtOAc (3×). The combined organics were dried (Na₂SO₄) and concentrated, and the residue was purified using column chromatography (silica gel, 0 to 60% EtOAc in hexanes) to afford methyl 3-allyl-6-chloro-1-isopropyl-1H-indole-4-carboxylate (550 mg, 67%) as a white solid. LC-MS(ES) m/z=292 [M+H]⁺.

### Preparation of 3-allyl-6-chloro-1-isopropyl-1H-indole-4-carboxylic acid

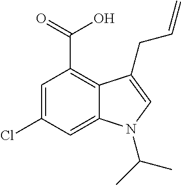 [SMILES: C=CCc1cn(C(C)C)c2cc(Cl)cc(C(=O)O)c12]

To a solution of methyl 3-allyl-6-chloro-1-isopropyl-1H-indole-4-carboxylate (500 mg, 1.714 mmol) in methanol (8 mL) was added aqueous sodium hydroxide (6 M, 1.428 mL, 8.57 mmol), and the mixture was stirred at room temperature for 18 hours. The mixture was concentrated to remove the methanol and the resulting aqueous solution was acidified using 1 N HCl to pH 3, then extracted with DCM (3×). The combined organics were dried over Na₂SO₄ and concentrated to afford 3-allyl-6-chloro-1-isopropyl-1H-indole-4-carboxylic acid (445 mg, 93%) as a white solid. LC-MS(ES) m/z=278 [M+H]⁺.

### Preparation of 3-allyl-N-((4-(but-3-en-1-yl)-2-methoxy-6-methylpyridin-3-yl)methyl)-6-chloro-1-isopropyl-1H-indole-4-carboxamide

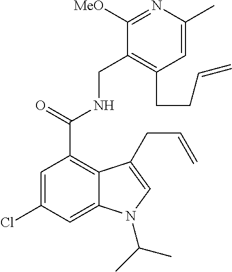 [SMILES: C=CCCc1cc(C)nc(OC)c1CNC(=O)c1cc(Cl)cc2c1c(CC=C)cn2C(C)C]

To a solution of 3-allyl-6-chloro-1-isopropyl-1H-indole-4-carboxylic acid (442 mg, 1.591 mmol) in dimethyl sulfoxide (8 mL) were added (4-(but-3-en-1-yl)-2-methoxy-6-methylpyridin-3-yl)methanamine (345 mg, 1.671 mmol), EDC (610 mg, 3.18 mmol), and N-methylmorpholine (0.875 mL, 7.96 mmol), and the mixture was stirred at room temperature for 18 hours. The mixture was quenched with water and extracted with EtOAc (3×). The combined organics were dried over Na₂SO₄ and concentrated. The residue was purified using column chromatography (silica gel, 0 to 100% EtOAc in hexanes) to afford 3-allyl-N-((4-(but-3-en-1-yl)-2-methoxy-6-methylpyridin-3-yl)methyl)-6-chloro-1-isopropyl-1H-indole-4-carboxamide (550 mg, 74%) as a white solid. LC-MS(ES) m/z=466 [M+H]⁺.

### Preparation of (E)-2-chloro-4-isopropyl-14-methoxy-12-methyl-9,10,15,16-tetrahydro-4H-pyrido[4′,3′:11,12][1]azacyclotridecino[5,4,3-cd]indol-17(6H)-one, and (Z)-2-chloro-4-isopropyl-14-methoxy-12-methyl-9,10,15,16-tetrahydro-4H-pyrido[4′,3′:11,12][1]azacyclotridecino[5,4,3-cd]indol-17(6H)-one

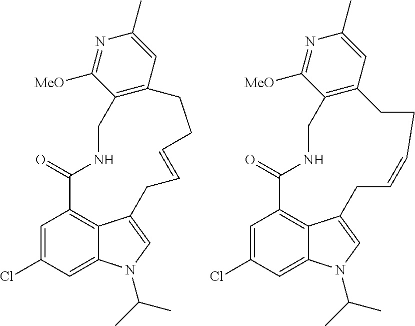 [SMILES: COc1nc(C)cc2c1CNC(=O)c1cc(Cl)cc3c1c(cn3C(C)C)C/C=C/CC2 ; COc1nc(C)cc2c1CNC(=O)c1cc(Cl)cc3c1c(cn3C(C)C)C/C=C\CC2]

To a solution of 3-allyl-N-((4-(but-3-en-1-yl)-2-methoxy-6-methylpyridin-3-yl)methyl)-6-chloro-1-isopropyl-1H-indole-4-carboxamide (200 mg, 0.429 mmol) in dichloromethane (5 mL) was added Grubbs second generation catalyst (36.4 mg, 0.043 mmol), and the mixture was stirred at reflux for 2 hours under a nitrogen atmosphere. The mixture was diluted and filtered. The filtrate was concentrated and the residue was purified using reverse-phase HPLC using Trilution software, with a phenomenex Gemini 5u C18(2) 100 A, AXIA 30×100 mm 5 micron, 10-minute run (30 mL/min, 50% ACN/H₂O, 0.1% TFA to 85% ACN/H₂O, 0.1% TFA) with UV detection at 254 nm to afford (E)-2-chloro-4-isopropyl-14-methoxy-12-methyl-9,10,15,16-tetrahydro-4H-pyrido[4′,3′:11,12][1]azacyclotridecino[5,4,3-cd]indol-17(6H)-one (46 mg, 24%) as a white solid. LC-MS(ES) m/z=438 [M+H]+.

Also isolated was (Z)-2-chloro-4-isopropyl-14-methoxy-12-methyl-9,10,15,16-tetrahydro-4H-pyrido[4′,3′:11,12][1]azacyclotridecino[5,4,3-cd]indol-17(6H)-one (21 mg, 11%) as a white solid LC-MS(ES) m/z=438 [M+H]⁺.

### Preparation of (E)-2-chloro-4-isopropyl-12-methyl-9,10,15,16-tetrahydro-4H-pyrido[4′,3′:11,12][1]azacyclotridecino[5,4,3-cd]indole-14,17(6H,13H)-dione

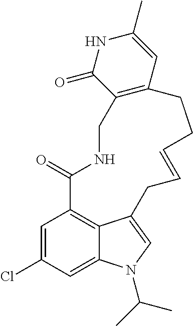 [SMILES: Cc1cc2c(c(=O)[nH]1)CNC(=O)c1cc(Cl)cc3c1c(cn3C(C)C)C/C=C/CC2]

To a solution of (E)-2-chloro-4-isopropyl-14-methoxy-12-methyl-9,10,15,16-tetrahydro-4H-benzo[11,12][1]azacyclotridecino[5,4,3-cd]indol-17(6H)-one (15 mg, 0.034 mmol) in 1,4-dioxane (2 mL) was added a solution of HCl in 1,4-dioxane (4 M, 500 μl, 2.000 mmol), and the mixture was stirred at 70° C. for 20 hours. The mixture was concentrated and the residue was purified using reverse-phase HPLC using Trilution software, with a phenomenex Gemini 5u C18(2) 100 A, AXIA 30×100 mm 5 micron, 10-minute run (30 mL/min, 40% ACN/H₂O, 0.1% formic acid to 80% ACN/H₂O, 0.1% formic acid) with UV detection at 254 nm to afford (E)-2-chloro-4-isopropyl-12-methyl-9,10,15,16-tetrahydro-4H-pyrido[4′,3′:11,12][1]azacyclotridecino[5,4,3-cd]indole-14,17(6H,13H)-dione (12 mg) as a white solid. LC-MS(ES) m/z=424 [M+H]⁺. ¹H NMR (600 MHz, DMSO-d₆) δ ppm 1.39 (d, J=6.80 Hz, 6H) 2.11 (s, 3H) 2.17 (br. s., 2H) 2.54 (br. s., 2H) 3.28 (br. s., 2H) 4.21 (br. s., 2H) 4.70-4.81 (m, 1H) 5.13 (br. s., 2H) 5.83 (s, 1H) 7.00 (s, 1H) 7.37 (s, 1H) 7.61-7.75 (m, 1H) 8.08 (br. s., 1H) 11.27 (s, 1H).

### Example 2

### (Z)-2-chloro-4-isopropyl-12-methyl-9,10,15,16-tetrahydro-4H-pyrido[4′,3′:11,12][1]azacyclotridecino[5,4,3-cd]indole-14,17(6H,13H)-dione

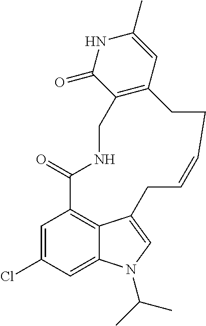 [SMILES: Cc1cc2c(c(=O)[nH]1)CNC(=O)c1cc(Cl)cc3c1c(cn3C(C)C)C/C=C\CC2]

To a solution of (Z)-2-chloro-4-isopropyl-14-methoxy-12-methyl-9,10,15,16-tetrahydro-4H-pyrido[4′,3′:11,12][1]azacyclotridecino[5,4,3-cd]indol-17(6H)-one (18 mg, 0.041 mmol) in 1,4-dioxane (2 mL) was added a solution of HCl in 1,4-dioxane (4 M, 500 μl, 2.000 mmol), and the mixture was stirred at 70° C. for 20 hours. The mixture was concentrated and the residue was washed with CH₃CN and dried under vacuum to afford (Z)-2-chloro-4-isopropyl-12-methyl-9,10,15,16-tetrahydro-4H-pyrido[4′,3′:11,12][1]azacyclotridecino[5,4,3-cd]indole-14,17(6H, 13H)-dione (17 mg) as an off-white solid. LC-MS(ES) m/z=424 [M+H]⁺. ¹H NMR (400 MHz, DMSO-d₆) δ ppm 1.44 (d J=7.05 Hz, 6H) 2.12 (s, 3H) 2.32-2.42 (m, 2H) 2.84 (t, J=6.95 Hz, 2H) 3.45 (d, J=5.56 Hz, 2H) 4.43 (d, J=5.31 Hz, 2H) 4.69-4.80 (m, 1H) 5.07-5.21 (m, 2H) 5.98 (s, 1H) 7.01 (d, J=1.52 Hz, 1H) 7.42 (s, 1H) 7.65 (d, J=1.77 Hz, 1H) 8.28 (t, J=5.43 Hz, 1H) 11.48 (br. s., 1H).

### Example 3

### 2-chloro-4-isopropyl-12-methyl-7,8,9,10,15,16-hexahydro-4H-pyrido[4′,3′:11,12][1]azacyclotridecino[5,4,3-cd]indole-14,17(6H,13H)-dione

### Preparation of 2-chloro-4-isopropyl-14-methoxy-12-methyl-7, 8,9,10,15,16-hexahydro-4H-pyrido[4′,3′:11,12][1]azacyclotridecino[5,4,3-cd]indol-17(6H)-one

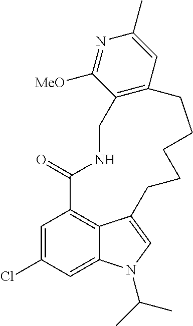 [SMILES: COc1nc(C)cc2c1CNC(=O)c1cc(Cl)cc3c1c(cn3C(C)C)CCCCC2]

A solution of (E)-2-chloro-4-isopropyl-14-methoxy-12-methyl-9,10,15,16-tetrahydro-4H-pyrido[4′,3′:11,12][1]azacyclotridecino[5,4,3-cd]indol-17(6H)-one (30 mg, 0.068 mmol) in ethyl acetate (2 mL) was degassed for 10 minutes by bubbling nitrogen. Pt/C (5 wt %, 8 mg) was added and the solution was purged with hydrogen for 5 minutes and then was stirred for 1 hour under a hydrogen atmosphere (balloon). The mixture was filtered and the filtrate was concentrated to afford 2-chloro-4-isopropyl-14-methoxy-12-methyl-7, 8,9,10,15,16-hexahydro-4H-pyrido[4′,3′: 11,12][1]azacyclotridecino[5,4,3-cd]indol-17(6H)-one (28 mg, 93%) as an off-white solid. LC-MS(ES) m/z=440 [M+H]⁺.

### Preparation of 2-chloro-4-isopropyl-12-methyl-7, 8,9,10,15,16-hexahydro-4H-pyrido[4′,3′:11,12][1]azacyclotridecino[5,4,3-cd]indole-14,17(6H,13H)-dione

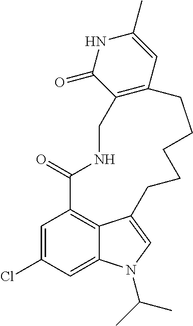 [SMILES: Cc1cc2c(c(=O)[nH]1)CNC(=O)c1cc(Cl)cc3c1c(cn3C(C)C)CCCCC2]

To a solution of 2-chloro-4-isopropyl-14-methoxy-12-methyl-7, 8,9,10,15,16-hexahydro-4H-pyrido[4′,3′:11,12][1]azacyclotridecino[5,4,3-cd]indol-17(6H)-one (26 mg, 0.059 mmol) in 1,4-dioxane (2 mL) was added a solution of HCl in 1,4-dioxane (4 M, 500 μl, 2.000 mmol), and the mixture was stirred at 70° C. for 20 hours. The mixture was concentrated and the residue was washed with CH₃CN and dried under vacuum to afford 2-chloro-4-isopropyl-12-methyl-7,8,9,10,15,16-hexahydro-4H-pyrido[4′,3′:11,12][1]azacyclotridecino[5,4,3-cd]indole-14,17(6H,13H)-dione (25 mg) as an off-white solid. LC-MS(ES) m/z=426 [M+H]⁺. ¹H NMR (400 MHz, DMSO-d₆) δ ppm 1.25 (br. s., 2H) 1.35-1.43 (m, 8H) 1.74-1.82 (m, 2H) 2.12 (s, 3H) 2.54-2.62 (m, 2H) 2.82 (t, J=6.57 Hz, 2H) 4.50 (d, J=5.56 Hz, 2H) 4.66-4.79 (m, 1H) 5.93 (s, 1H) 6.97 (d, J=1.77 Hz, 1H) 7.34 (s, 1H) 7.62 (d, J=1.77 Hz, 1H) 8.54 (t, J=5.56 Hz, 1H) 11.46 (s, 1H)

### Example 4

### (E)-4-isopropyl-12-methyl-2-(6-(piperazin-1-yl)pyridin-3-yl)-9,10,15,16-tetrahydro-4H-pyrido[4′,3′:11,12][1]azacyclotridecino[5,4,3-cd]indole-14,17(6H,13H)-dione

### Preparation of methyl 3-allyl-6-bromo-1H-indole-4-carboxylate

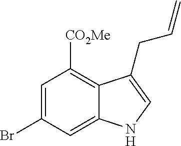 [SMILES: C=CCc1c[nH]c2cc(Br)cc(C(=O)OC)c12]

To a solution of methyl 6-bromo-1H-indole-4-carboxylate (700 mg, 2.76 mmol) in THF (12 mL) was added allyl alcohol (0.187 mL, 2.76 mmol), and the mixture was degassed by sparging with nitrogen for 5 minutes. Pd(PPh₃)₄ (159 mg, 0.138 mmol) and triethylborane (1 M, THF, 0.827 mL, 0.827 mmol) were added and the mixture was heated to 75° C. and stirred for 7 hours. The mixture was concentrated and the residue was purified using column chromatography (silica gel, 0 to 60% EtOAc in hexanes) to afford methyl 3-allyl-6-bromo-1H-indole-4-carboxylate (535 mg, 66%) as a white solid. LC-MS(ES) m/z=293.9, 295.9 [M+H]+.

### Preparation of methyl 3-allyl-6-bromo-1-isopropyl-1H-indole-4-carboxylate

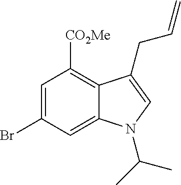 [SMILES: C=CCc1cn(C(C)C)c2cc(Br)cc(C(=O)OC)c12]

To a solution of methyl 3-allyl-6-bromo-1H-indole-4-carboxylate (530 mg, 1.802 mmol) in DMF (10 mL) was added NaH (60% dispersion in mineral oil, 108 mg, 2.70 mmol), and the mixture was stirred at 0° C. for 30 min. 2-Iodopropane (0.234 mL, 2.342 mmol) was added and the reaction mixture was stirred at room temperature for 18 hours. The mixture was quenched with 10% aqueous NH₄Cl solution and extracted with EtOAc (3×). The combined organics were dried over Na₂SO₄ and concentrated. The residue was purified using column chromatography (silica gel, 0 to 60% EtOAc in hexanes) to afford methyl 3-allyl-6-bromo-1-isopropyl-1H-indole-4-carboxylate (440 mg, 73%) as a pale yellow oil. LC-MS(ES) m/z=336.0, 338.1 [M+H]⁺.

### Preparation of 3-allyl-6-bromo-1-isopropyl-1H-indole-4-carboxylic acid

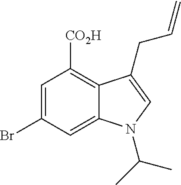 [SMILES: C=CCc1cn(C(C)C)c2cc(Br)cc(C(=O)O)c12]

To a solution of methyl 3-allyl-6-bromo-1-isopropyl-1H-indole-4-carboxylate (440 mg, 1.309 mmol) in MeOH (6 mL) was added aqueous NaOH (5 M, 1.091 mL, 6.54 mmol), and the mixture was stirred at room temperature for 18 hours. The mixture was concentrated to remove MeOH and the resulting aqueous solution was acidified using aqueous HCl (1 N) to pH 3, then extracted with DCM (3×). The combined organics were dried over Na₂SO₄ and concentrated to afford 3-allyl-6-bromo-1-isopropyl-1H-indole-4-carboxylic acid (395 mg, 94%) as a pale yellow solid. LC-MS(ES) m/z=322.0, 324.0 [M+H]⁺.

### Preparation of 3-allyl-6-bromo-N-((4-(but-3-en-1-yl)-2-methoxy-6-methylpyridin-3-yl)methyl)-1-isopropyl-1H-indole-4-carboxamide

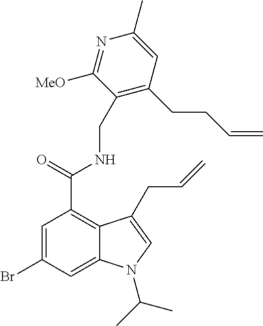 [SMILES: C=CCCc1cc(C)nc(OC)c1CNC(=O)c1cc(Br)cc2c1c(CC=C)cn2C(C)C]

To a solution of 3-allyl-6-bromo-1-isopropyl-1H-indole-4-carboxylic acid (390 mg, 1.210 mmol) in DMSO (6 mL) were added (4-(but-3-en-1-yl)-2-methoxy-6-methylpyridin-3-yl)methanamine (250 mg, 1.210 mmol), N-methylmorpholine (0.798 mL, 7.26 mmol), EDC (464 mg, 2.421 mmol), and HOAt (330 mg, 2.421 mmol), and the mixture was stirred at room temperature for 18 hours. The mixture was quenched with water and extracted with EtOAc (3×). The combined organics were dried over Na₂SO₄ and concentrated. The residue was purified using column chromatography (silica gel, 0 to 100% EtOAc in hexanes) to afford 3-allyl-6-bromo-N-((4-(but-3-en-1-yl)-2-methoxy-6-methylpyridin-3-yl)methyl)-1-isopropyl-1H-indole-4-carboxamide (530 mg, 86%) as an off-white solid. LC-MS(ES) m/z=510.2, 512.2 [M+H]⁺.

### Preparation of (E)-2-bromo-4-isopropyl-14-methoxy-12-methyl-9,10,15,16-tetrahydro-4H-pyrido[4′,3′:11,12][1]azacyclotridecino[5,4,3-cd]indol-17(6H)-one

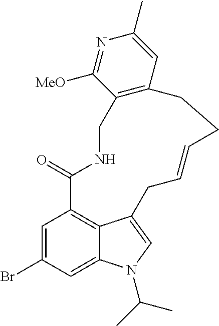 [SMILES: COc1nc(C)cc2c1CNC(=O)c1cc(Br)cc3c1c(cn3C(C)C)C/C=C/CC2]

A solution of 3-allyl-6-bromo-N-((4-(but-3-en-1-yl)-2-methoxy-6-methylpyridin-3-yl)methyl)-1-isopropyl-1H-indole-4-carboxamide (360 mg, 0.705 mmol) in DCM (30 mL) was degassed by sparging with nitrogen for 5 minutes. Grubbs first generation catalyst (32 mg, 0.038 mmol) was added and the reaction mixture was stirred at room temperature for 7 hours. The mixture was concentrated and the residue was purified using column chromatography (silica gel, 0 to 80% EtOAc in hexanes) to afford (E)-2-bromo-4-isopropyl-14-methoxy-12-methyl-9,10,15,16-tetrahydro-4H-pyrido[4′,3′: 11,12][1]azacyclotridecino[5,4,3-cd]indol-17(6H)-one (178 mg, 52%) as a white solid. LC-MS(ES) m/z=482.1, 484.1 [M+H]⁺.

### Preparation of (E)-tert-butyl 4-(5-(4-isopropyl-14-methoxy-12-methyl-17-oxo-6,9,10,15,16,17-hexahydro-4H-pyrido[4′,3′: 11,12][1]azacyclotridecino[5,4,3-cd]indol-2-yl)pyridin-2-yl)piperazine-1-carboxylate

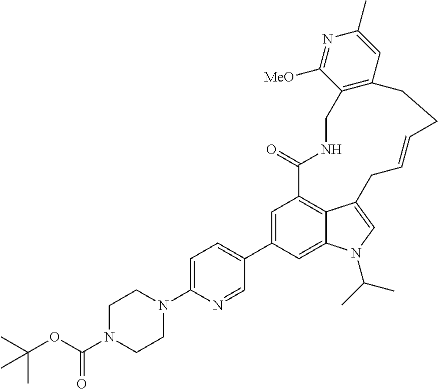 [SMILES: COc1nc(C)cc2c1CNC(=O)c1cc(-c3ccc(N4CCN(C(=O)OC(C)(C)C)CC4)nc3)cc3c1c(cn3C(C)C)C/C=C/CC2]

To a 10 mL microwave tube were added (E)-2-bromo-4-isopropyl-14-methoxy-12-methyl-9,10,15,16-tetrahydro-4H-pyrido[4′,3′:11,12][1]azacyclotridecino[5,4,3-cd]indol-17(6H)-one (80 mg, 0.166 mmol), tert-butyl 4-(5-(4,4,5,5-tetramethyl-1,3,2-dioxaborolan-2-yl)pyridin-2-yl)piperazine-1-carboxylate (84 mg, 0.216 mmol), DME (2 mL), water (0.667 mL) and aqueous Na₂CO₃ (2 M, 0.083 mL, 0.166 mmol), and the mixture was degassed by sparging with nitrogen for 5 minutes. PdCl₂(dppf)-DCM (10.83 mg, 0.013 mmol) was added and the tube was sealed. The mixture was heated at 140° C. for 20 min in a microwave reactor. The mixture was concentrated and the residue was taken up into MeOH and filtered. The filtrate was purified using reverse-phase HPLC using Trilution software, with a Gemini 5u C18 110 A, AXIA 30×50 mm 5 micron 10-minute run (30 mL/min, 45% ACN/H₂O, 0.1% NH₄OH to 80% ACN/H₂O, 0.1% NH₄OH) with UV detection at 254 nm to afford (E)-tert-butyl 4-(5-(4-isopropyl-14-methoxy-12-methyl-17-oxo-6,9,10,15,16,17-hexahydro-4H-pyrido[4′,3′:11,12][1]azacyclotridecino[5,4,3-cd]indol-2-yl)pyridin-2-yl)piperazine-1-carboxylate (67 mg. 61%) as an off-white solid. LC-MS(ES) m/z=665.5 [M+H]⁺ (minor), 305.3 (major).

### Preparation of (E)-4-isopropyl-12-methyl-2-(6-(piperazin-1-yl)pyridin-3-yl)-9,10,15,16-tetrahydro-4H-pyrido[4′,3′:11,12][1]azacyclotridecino[5,4,3-cd]indole-14,17(6H,13H)-dione dihydrochloride

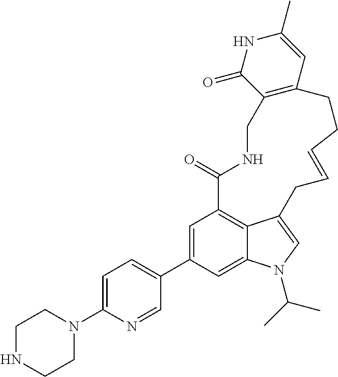 [SMILES: Cc1cc2c(c(=O)[nH]1)CNC(=O)c1cc(-c3ccc(N4CCNCC4)nc3)cc3c1c(cn3C(C)C)C/C=C/CC2]

To a solution of (E)-tert-butyl 4-(5-(4-isopropyl-14-methoxy-12-methyl-17-oxo-6,9,10,15,16,17-hexahydro-4H-pyrido[4′,3′:11,12][1]azacyclotridecino[5,4,3-cd]indol-2-yl)pyridin-2-yl)piperazine-1-carboxylate (64 mg, 0.096 mmol) in 1,4-dioxane (3 mL) was added HCl (4 M in 1,4-dioxane, 0.6 mL, 2.400 mmol), and the mixture was stirred at 70° C. for 20 hours. The mixture was concentrated and the residue was purified using reverse-phase HPLC using Trilution software, with a phenomenex Gemini 5u C18(2) 100 A, AXIA 30×100 mm 5 micron 10-minute run (30 mL/min, 20% ACN/H₂O, 0.1% formic acid to 80% ACN/H₂O, 0.1% formic acid) with UV detection at 254 nm to afford (E)-4-isopropyl-12-methyl-2-(6-(piperazin-1-yl)pyridin-3-yl)-9,10,15,16-tetrahydro-4H-pyrido[4′,3′:11,12][1]azacyclotridecino[5,4,3-cd]indole-14,17(6H,13H)-dione dihydrochloride (39 mg, 64%) as an off-white solid. LC-MS(ES) m/z=551.3 [M+H]⁺ (minor), 276.2 (major). ¹H NMR (400 MHz, DMSO-d₆) δ: 1.44 (d, J=6.4 Hz, 6H), 2.15 (s, 3H), 2.17-2.25 (m, 2H), 2.54-2.64 (m, 3H), 3.22-3.37 (m, 6H), 3.88-4.02 (m, 5H), 4.20-4.34 (m, 1H), 4.85-4.96 (m, 1H), 5.10-5.23 (m, 2H), 5.94 (s, 1H), 7.28-7.36 (m, 2H), 7.39 (s, 1H), 7.89 (s, 1H), 8.16-8.20 (m, 1H), 8.28-8.40 (m, 1H), 8.50-8.53 (m, 1H), 9.44 (br. s., 2H), 11.49 (br. s., 1H).

### Example 5

### (E)-4-isopropyl-12-methyl-14,17-dioxo-6,9,10,13,14,15,16,17-octahydro-4H-pyrido[4′,3′:11,12][1]azacyclotridecino[5,4,3-cd]indole-2-carbonitrile

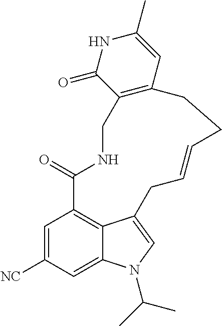 [SMILES: Cc1cc2c(c(=O)[nH]1)CNC(=O)c1cc(C#N)cc3c1c(cn3C(C)C)C/C=C/CC2]

To a 10 mL sealable tube were added (E)-2-bromo-4-isopropyl-12-methyl-9,10,15,16-tetrahydro-4H-pyrido[4′,3′:11,12][1]azacyclotridecino[5,4,3-cd]indole-14,17(6H,13H)-dione (50 mg, 0.107 mmol), dicyanozinc (14.41 mg, 0.123 mmol), DMF (1 mL), dppf (7.69 mg, 0.014 mmol), zinc (1.745 mg, 0.027 mmol), and Pd₂(dba)₃ (12.71 mg, 0.014 mmol), and the mixture was degassed by sparging with nitrogen. The tube was sealed and the mixture was heated at 120° C. for 6 hours. The mixture was concentrated and the residue was treated with DMSO and filtered. The filtrate was purified using reverse-phase HPLC using Trilution software, with a phenomenex Gemini 5u C18(2) 100 A, AXIA 30×100 mm 5 micron 10-minute run (30 mL/min, 38% ACN/H₂O, 0.1% formic acid to 78% ACN/H₂O, 0.1% formic acid) with UV detection at 254 nm to afford (E)-4-isopropyl-12-methyl-14,17-dioxo-6,9,10,13,14,15,16,17-octahydro-4H-pyrido[4′,3′:11,12][1]azacyclotridecino[5,4,3-cd]indole-2-carbonitrile (15 mg, 33%) of product as an off-white solid. LC-MS(ES) m/z=415.2 [M+H]⁺. ¹H NMR (400 MHz, DMSO-d₆) δ: 1.43 (d, J=6.6 Hz, 6H), 2.12 (s, 3H), 2.18 (br. s., 2H), 2.54-2.62 (m, 2H), 3.30-3.33 (m, 2H), 4.09-4.35 (m, 2H), 4.87 (quin., J=6.6 Hz, 1H), 5.03-5.21 (m, 2H), 5.84 (s, 1H), 7.32 (d, J=1.3 Hz, 1H), 7.68 (s, 1H), 8.21 (d, J=1.3 Hz, 2H), 11.29 (s, 1H).

### Example 6

### (E)-2-(3-hydroxy-3-methylbut-1-yn-1-yl)-4-isopropyl-12-methyl-9,10,15,16-tetrahydro-4H-pyrido[4′,3′:11,12][1]azacyclotridecino[5,4,3-cd]indole-14,17(6H,13H)-dione

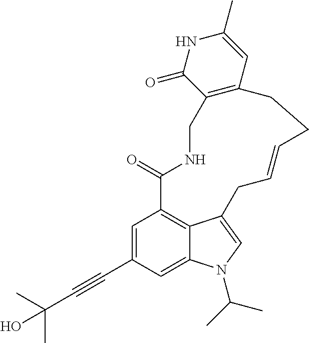 [SMILES: Cc1cc2c(c(=O)[nH]1)CNC(=O)c1cc(C#CC(C)(C)O)cc3c1c(cn3C(C)C)C/C=C/CC2]

To a 10 mL microwave tube were added (E)-2-bromo-4-isopropyl-12-methyl-9,10,15,16-tetrahydro-4H-pyrido[4′,3′:11,12][1]azacyclotridecino[5,4,3-cd]indole-14,17(6H,13H)-dione, (70 mg, 0.090 mmol), NaI (2.69 mg, 0.018 mmol), zinc (1.172 mg, 0.018 mmol), DMSO (2 mL), and DBU (0.014 mL, 0.090 mmol), and the mixture was degassed by sparging with nitrogen for 5 minutes. 2-Methylbut-3-yn-2-ol (37.7 mg, 0.448 mmol) and Pd(PPh₃)₄ (10.36 mg, 8.97 μmol) were added and the tube was sealed. The mixture was stirred at 85° C. for 3 hours. The mixture was cooled and filtered. The filtrate was purified using reverse-phase HPLC using Trilution software, with a phenomenex Gemini 5u C18(2) 100 A, AXIA 30×100 mm 5 micron, 10-minute run (30 mL/min, 35% ACN/H₂O, 0.1% formic acid to 70% ACN/H₂O, 0.1% formic acid) with UV detection at 254 nm to afford (E)-2-(3-hydroxy-3-methylbut-1-yn-1-yl)-4-isopropyl-12-methyl-9,10,15,16-tetrahydro-4H-pyrido[4′,3′:11,12][1]azacyclotridecino[5,4,3-cd]indole-14,17(6H,13H)-dione (13 mg, 30%) as an off-white solid. LC-MS(ES) m/z=472.3 [M+H]⁺. ¹H NMR (400 MHz, DMSO-d₆) δ: 1.40 (d, J=6.8 Hz, 6H), 1.50 (s, 6H), 2.11 (s, 3H), 2.18 (br. s., 2H), 2.55 (br. s., 2H), 3.29 (br. s., 2H), 4.22 (br. s., 2H), 4.78 (quin., J=6.6 Hz, 1H), 5.08-5.22 (m, 2H), 5.46 (s, 1H), 5.84 (s, 1H), 7.01 (d, J=1.0 Hz, 1H), 7.41 (s, 1H), 7.60 (d, J=1.3 Hz, 1H), 8.01 (br. s., 1H), 11.30 (s, 1H).

### Assay Protocol

Compounds contained herein were evaluated for their ability to inhibit the methyltransferase activity of EZH2 within the PRC2 complex. Human PRC2 complex was prepared by co-expressing each of the 5 member proteins (FLAG-EZH2, EED, SUZ12, RbAp48, AEBP2) in Sf9 cells followed by co-purification. Enzyme activity was measured in a scintillation proximity assay (SPA) where a tritiated methyl group is transferred from 3H-SAM to a lysine residue on Histone H3 of a mononucleosome, purified from HeLa cells. Mononucleosomes were captured on SPA beads and the resulting signal is read on a ViewLux plate reader.

### Part A. Compound Preparation

1. Prepare 10 mM stock of compounds from solid in 100% DMSO.
2. Set up an 11-point serial dilution (1:3 dilution, top concentration 10 mM) in 100% DMSO for each test compound in a 384 well plate leaving columns 6 and 18 for DMSO controls.
3. Dispense 100 nL of compound from the dilution plate into reaction plates (Grenier Bio-One, 384-well, Cat#784075).

### Part B. Reagent Preparation

### Prepare the Following Solutions:

1. 50 mM Tris-HCl, pH 8: Per 1 L of base buffer, combine 1 M Tris-HCl, pH 8 (50 mL) and distilled water (950 mL).
2. 1× Assay Buffer: Per 10 mL of 1× Assay Buffer, combine 50 mM Tris-HCl, pH 8 (9958 uL), 1 M MgCl₂ (20 uL), 2 M DTT (20 uL), and 10% Tween-20 (2 uL) to provide a final concentration of 50 mM Tris-HCl, pH 8, 2 mM MgCl₂, 4 mM DTT, 0.002% Tween-20.
3. 2× Enzyme Solution: Per 10 mL of 2× Enzyme Solution, combine 1× Assay Buffer and PRC2 complex to provide a final enzyme concentration of 10 nM.
4. SPA Bead Suspension: Per 1 mL of SPA Bead Suspension, combine PS-PEI coated LEADSeeker beads (40 mg) and ddH₂O (1 mL) to provide a final concentration of 40 mg/mL.
5. 2× Substrate Solution: Per 10 mL of 2× Substrate Solution, combine 1× Assay Buffer (9728.55 uL), 800 ug/mL mononucleosomes (125 uL), 1 mM cold SAM (4 uL), and 7.02 uM 3H-SAM (142.45 uL; 0.55 mCi/mL) to provide a final concentration of 5 ug/mL nucleosomes, 0.2 uM cold SAM, and 0.05 uM 3H-SAM.
6. 2.67× Quench/Bead Mixture: Per 10 mL of 2.67× Quench/Bead Mixture, combine ddH₂O (9358 uL), 10 mM cold SAM (267 uL), 40 mg/mL Bead Suspension (375 uL) to provide a final concentration of 100 uM cold SAM and 0.5 mg/mL SPA beads.

### Part C. Assay Reaction in 384-Well Grenier Bio-One Plates

### Compound Addition

1. Dispense 100 nL/well of 100× Compound to test wells (as noted above).
2. Dispense 100 nL/well of 100% DMSO to columns 6 & 18 for high and low controls, respectively.

### Assay

1. Dispense 5 uL/well of 1× Assay Buffer to column 18 (low control reactions).
2. Dispense 5 uL/well of 2× Enzyme Solution to columns 1-17, 19-24.
3. Spin assay plates for −1 minute at 500 rpm.
4. Stack the assay plates, covering the top plate.
5. Incubate the compound/DMSO with the enzyme for 30 minutes at room temperature.
6. Dispense 5 uL/well of 2× Substrate Solution to columns 1-24.
7. Spin assay plates for −1 minute at 500 rpm.
8. Stack the assay plates, covering the top plate.
9. Incubate the assay plates at room temperature for 1 hour.

### Quench/Bead Addition

1. Dispense 5 uL/well of the 3× Quench/Bead Mixture to columns 1-24.
2. Seal the top of each assay plate with adhesive TopSeal.
3. Spin assay plates for 1 minute at 500 rpm.
4. Equilibrate the plates for >20 min.

### Read Plates

1. Read the assay plates on the Viewlux Plate Reader utilizing the 613 nm emission filter with a 300 s read time.
   Reagent addition can be done manually or with automated liquid handler.
   *The final DMSO concentration in this assay is 1%.
   *The positive control is in column 6; negative control is in column 18.
   *Final starting concentration of compounds is 100 M.

### Results

Percent inhibition was calculated relative to the DMSO control for each compound concentration and the resulting values were fit using standard IC₅₀ fitting parameters within the ABASE data fitting software package.

Exemplified compounds of the present invention were generally tested according to the above or an analogous assay and were found to be inhibitors of EZH2. The IC₅₀ values ranged from about 40 nM to about 500 nM. Specific biological activities tested according to assays described herein are listed in the following table. Repeating the assay run(s) may result in somewhat different IC₅₀ values.

| Example | EZH2 IC₅₀ (nM) |
|---|---|
| 1 | 40 |
| 2 | 316 |
| 3 | 500 |
| 4 | 32 |
| 5 | 158 |
| 6 | 200 |

1. A compound according to Formula (I): 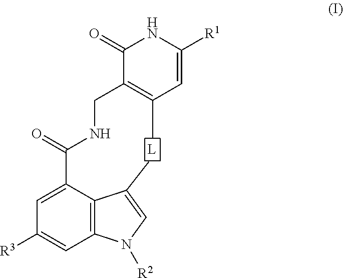
wherein:
L is (C₂-C₈)alkylenyl or (C₂-C₈)alkenylenyl;
R¹ is hydrogen, halogen, (C₁-C₆)alkyl, (C₂-C₆)alkenyl, (C₂-C₆)alkynyl, halo(C₁-C₄)alkyl, (C₃-C₆)cycloalkyl, (C₃-C₆)cycloalkyl(C₁-C₆)alkyl, (C₃-C₆)cycloalkyl(C₂-C₆)alkenyl, (C₅-C₆)cycloalkenyl, (C₅-C₆)cycloalkenyl(C₁-C₆)alkyl, (C₅-C₆)cycloalkenyl(C₂-C₆)alkenyl, (C₆-C₁₀)bicycloalkyl, heterocycloalkyl, heterocycloalkyl(C₁-C₆)alkyl-, heterocycloalkyl(C₂-C₆)alkenyl, phenyl, phenyl(C₁-C₆)alkyl, phenyl(C₂-C₆)alkenyl, heteroaryl, heteroaryl(C₁-C₆)alkyl, heteroaryl(C₂-C₆)alkenyl, cyano, —C(O)R^{a}, —CO₂R^{a}, —C(O)NR^{a}R^{b}, —C(O)NR^{a}NR^{a}R^{b}, —SR^{a}, —S(O)R^{a}, —SO₂R^{a}, —SO₂NR^{a}R^{b}, nitro, —NR^{a}R^{b}, R^{a}R^{b}N(C₁-C₄)alkyl-, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, or —OC(O)NR^{a}R^{b}, wherein each cycloalkyl, cycloalkenyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂N^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;
R² is hydrogen, (C₁-C₆)alkyl, (C₂-C₆)alkenyl, (C₂-C₆)alkynyl, halo(C₁-C₄)alkyl, (C₃-C₆)cycloalkyl, (C₃-C₆)cycloalkyl(C₁-C₆)alkyl, (C₃-C₆)cycloalkyl(C₂-C₆)alkenyl, (C₅-C₆)cycloalkenyl, (C₅-C₆)cycloalkenyl(C₁-C₆)alkyl, (C₅-C₆)cycloalkenyl(C₂-C₆)alkenyl, (C₆-C₁₀)bicycloalkyl, heterocycloalkyl, heterocycloalkyl(C₁-C₆)alkyl-, heterocycloalkyl(C₂-C₆)alkenyl, phenyl, phenyl(C₁-C₆)alkyl, phenyl(C₂-C₆)alkenyl, heteroaryl, heteroaryl(C₁-C₆)alkyl, heteroaryl(C₂-C₆)alkenyl, —C(O)R^{a}, —CO₂R^{a}, —C(O)NR^{a}R^{b}, or —C(O)NR^{a}NR^{a}R^{b}, wherein each cycloalkyl, cycloalkenyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;
R³ is selected from the group consisting of hydrogen, halogen, (C₁-C₆)alkyl, (C₂-C₆)alkenyl, (C₂-C₆)alkynyl, (C₁-C₄)alkoxy, —B(OH)₂, (C₃-C₆)cycloalkyl, (C₃-C₆)cycloalkyl(C₁-C₄)alkyl-, (C₆-C₁₀)bicycloalkyl, heterocycloalkyl, heterocycloalkyl(C₁-C₄)alkyl-, phenyl, phenyl(C₁-C₂)alkyl, heteroaryl, heteroaryl(C₁-C₂)alkyl, cyano, —C(O)R^{a}, —CO₂R^{a}, —C(O)NR^{a}R^{b}, —C(O)NR^{a}NR^{a}R^{b}, —SR^{a}, —S(O)R^{a}, —SO₂R^{a}, —SO₂NR^{a}R^{b}, nitro, —NR^{a}R^{b}, R^{a}R^{b}N(C₁-C₄)alkyl-, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, R^{a}O(C₁-C₄)alkyl-, R^{a}O(C₃-C₆)alkynyl-, —OC(O)R^{a}, and —OC(O)NR^{a}R^{b}, wherein each cycloalkyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;
each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and
R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl (C₁-C₂)alkyl-, heteroaryl(C₁-C₄)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂;
or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring;
or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring;
or a pharmaceutically acceptable salt thereof.

2. The compound or pharmaceutically acceptable salt according to claim 1, wherein R¹ is hydrogen, halogen, (C₁-C₆)alkyl, halo(C₁-C₄)alkyl, (C₃-C₆)cycloalkyl, (C₃-C₆)cycloalkyl(C₁-C₄)alkyl, phenyl, or phenyl(C₁-C₂)alkyl.

3. The compound or pharmaceutically acceptable salt according to claim 2, wherein R¹ is (C₁-C₄)alkyl.

4. The compound or pharmaceutically acceptable salt according to claim 1, wherein R² is hydrogen, (C₁-C₆)alkyl, halo(C₁-C₄)alkyl, (C₃-C₆)cycloalkyl, (C₃-C₆)cycloalkyl(C₁-C₄)alkyl, phenyl, or phenyl(C₁-C₂)alkyl.

5. The compound or pharmaceutically acceptable salt according to claim 4, wherein R² is (C₁-C₄)alkyl.

6. The compound or pharmaceutically acceptable salt according to claim 1, wherein R³ is halogen.

7. The compound or pharmaceutically acceptable salt according to claim 1, wherein R³ is heteroaryl which is optionally substituted 1 or 2 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;
each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and
R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl(C₁-C₂)alkyl-, heteroaryl(C₁-C₂)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂;
or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring;
or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring.

8. The compound or pharmaceutically acceptable salt according to claim 7, wherein R³ is pyridinyl which is optionally substituted by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;
each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and
R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl(C₁-C₂)alkyl-, heteroaryl(C₁-C₂)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂;
or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring;
or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring.

9. The compound or pharmaceutically acceptable salt according to claim 8, wherein R³ is pyridinyl which is optionally substituted by heterocycloalkyl or (C₁-C₄)alkyl-heterocycloalkyl-.

10. The compound or pharmaceutically acceptable salt according to claim 1, wherein L is (C₄-C₅)alkylenyl or (C₄-C₅)alkenylenyl.

11. The compound or pharmaceutically acceptable salt according to claim 10, wherein L is selected from the group consisting of:

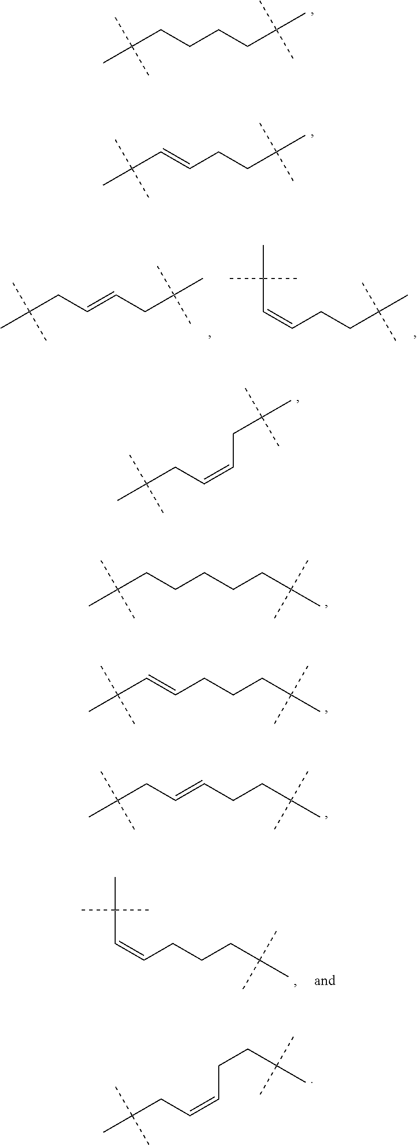

12. The compound according to claim 1 which is:
(E)-2-chloro-4-isopropyl-12-methyl-9,10,15,16-tetrahydro-4H-pyrido[4′,3′:11,12][1]azacyclotridecino[5,4,3-cd]indole-14,17(6H,13H)-dione;
(Z)-2-chloro-4-isopropyl-12-methyl-9,10,15,16-tetrahydro-4H-pyrido[4′,3′:11,12][1]azacyclotridecino[5,4,3-cd]indole-14,17(6H,13H)-dione;
2-chloro-4-isopropyl-12-methyl-7,8,9,10,15,16-hexahydro-4H-pyrido[4′,3′:11,12][1]azacyclotridecino[5,4,3-cd]indole-14,17(6H,13H)-dione;
(E)-4-isopropyl-12-methyl-2-(6-(piperazin-1-yl)pyridin-3-yl)-9,10,15,16-tetrahydro-4H-pyrido[4′,3′:11,12][1]azacyclotridecino[5,4,3-cd]indole-14,17(6H,13H)-dione;
(E)-4-isopropyl-12-methyl-14,17-dioxo-6,9,10,13,14,15,16,17-octahydro-4H-pyrido[4′,3′:11,12][1]azacyclotridecino[5,4,3-cd]indole-2-carbonitrile; or
(E)-2-(3-hydroxy-3-methylbut-1-yn-1-yl)-4-isopropyl-12-methyl-9,10,15,16-tetrahydro-4H-pyrido[4′,3′:11,12][1]azacyclotridecino[5,4,3-cd]indole-14,17(6H,13H)-dione;
or a pharmaceutically acceptable salt thereof.

13. A pharmaceutical composition comprising the compound or pharmaceutically acceptable salt thereof according to claim 1 and a pharmaceutically acceptable excipient.

14. A method of treating cancer comprising administering to a patient with cancer a therapeutically effective amount of the compound or pharmaceutically acceptable salt thereof according to claim 1.

15. The method of claim 14, wherein said cancer is selected from the group consisting of: brain, glioblastomas, leukemias, lymphomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, gastric, bladder, head and neck, kidney, lung, liver, melanoma, renal, ovarian, pancreatic, prostate, sarcoma, osteosarcoma, giant cell tumor of bone, and thyroid.

16. (canceled)
